(19) United States
(12) Patent Application Publication
(10) Pub. No.: US 2008/0154230 A1
(43) Pub. Date: Jun. 26, 2008

(54) ANODE FOR ELECTROTRANSPORT OF CATIONIC DRUG

(76) Inventors: Janardhanan Anand Subramony, Andhra Pradesh (IN); Yoshiko Katori, Oakland, CA (US); Rama Padmanabhan, Los Altos, CA (US); Joseph Bradley Phipps, Sunnyvale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/958,522

(22) Filed: Dec. 18, 2007

Related U.S. Application Data
(60) Provisional application No. 60/871,086, filed on Dec. 20, 2006.
(60) Provisional application No. 60/916,501, filed on May 7, 2007.
(60) Provisional application No. 60/981,877, filed on Oct. 23, 2007.

(51) Int. Cl. A61N 1/30; B65D 69/00

(52) U.S. Cl. 604/501; 206/570

(57) ABSTRACT

An electrotransport system for delivery of an electrotransport cationic drug. The system has an anode that has a precipitating anion source. The precipitating anions from the precipitating anion source combines with metal ions generated from sacrificial metal of the anode during electrotransport to form precipitates. Metal that can form the metal ions are embedded in the anode.

### CROSS REFERENCE TO RELATED U.S. APPLICATION DATA

The present application is derived from and claims priority to provisional applications U.S. Ser. No. 60/871,086, filed Dec. 20, 2006; U.S. Ser. No. 60/916,501, filed May 7, 2007; and U.S. Ser. No. 60/981,877, filed Oct. 23, 2007, which are herein incorporated by reference in their entireties.

### TECHNICAL FIELD

The present invention relates to an electrotransport drug delivery system having an anode for driving cationic drugs across a body surface or membrane. In particular, the invention relates to a system having an anode for transdermal administration of cationic drugs across a body surface or membrane by electrotransport such that the electrotransport does not cause staining on the body surface.

### BACKGROUND

The delivery of active agents through the skin provides many advantages, including comfort, convenience, and non-invasiveness. Gastrointestinal irritation and the variable rates of absorption and metabolism including first pass effect encountered in oral delivery are avoided. Transdermal delivery also provides a high degree of control over blood concentrations of any particular active agent.

Many active agents are not suitable for passive transdermal delivery because of their size, ionic charge characteristics, and hydrophilicity. One method for transdermal delivery of such active agents involves the use of electrical current to transport actively the active agent into the body through a body surface (e.g., intact skin) by electrotransport. Electrotransport techniques may include iontophoresis, electroosmosis, and electroporation. Electrotransport devices, such as iontophoretic devices are known in the art, e.g., U.S. Pat. Nos. 5,057,072; 5,084,008; 5,147,297; 5,395,310; 5,503,632; 5,871,461; 6,039,977; 6,049,733; 6,181,963, 6,216,033, 6,881,208, and US Patent Publications 20020128591, 20030191946, 20060089591, 20060173401, 20060241548. One electrode, called the active or donor electrode, is the electrode from which the active agent is delivered into the body. The other electrode, called the counter or return electrode, serves to close the electrical circuit through the body. In conjunction with the patient's body tissue, e.g., skin, the circuit is completed by connection of the electrodes to a source of electrical energy, and usually to circuitry capable of controlling the current passing through the device. If the ionic substance to be driven into the body is positively charged, then the positive electrode (the anode) will be the active electrode and the negative electrode (the cathode) will serve as the counter electrode. If the ionic substance to be delivered is negatively charged, then the cathodic electrode will be the active electrode and the anodic electrode will be the counter electrode.

Electrotransport devices require a reservoir or source of the active agent that is to be delivered or introduced into the body. Such reservoirs are connected to the anode or the cathode of the electrotransport device to provide a fixed or renewable source of one or more desired active agents. As electrical current flows through an electrotransport device, oxidation of a chemical species takes place at the anode while reduction of a chemical species takes place at the cathode. Typically, both of these reactions can generate a mobile ionic species with a charge state like that of the active agent in its ionic form. Such mobile ionic species are referred to as competitive species or competitive ions because the species can potentially compete with the active agent for delivery by electrotransport. For example, silver ions generated at the anode can compete with a cationic drug, and chloride ions formed at the cathode can compete with an anionic drug.

In electrotransport or iontophoretic technology, typically, consumable Ag and AgCl electrodes are used at the anode and cathode respectively. The use of consumable electrodes as opposed to the non-consumable platinum or stainless steel electrode has the advantage of mitigating pH shifts induced at the electrode-formulation interface due to electrolysis of water with the latter even at very low voltages.

At the silver anode, during electrotransport, silver is oxidized and, as a result, sliver ion is generated. At the cathode, typically AgCl (solid) is reduced to form metallic silver and chloride ion.

Ag→Ag⁺+e⁻

AgCl(s)+e⁻→Ag^{o}(s)+Cl⁻

At the anode, if silver ions are left to migrate, they can compete with the cationic drug to be delivered and reduce its transport efficiency. Furthermore, silver when allowed to migrate into the tissue of the patient results in a stain on the tissue, which is unsightly. Although the formulation of a cationic drug reservoir with a hydrochloride salt of the drug helps to precipitate some of the silver ions formed in the electrotransport as insoluble AgCl, an excess of the HCl drug salt is needed to ensure that enough chloride is available for interface electrochemistry and to maintain steady state delivery without depletion. However, excessive drug loading could be costly and would increase the potential for drug abuse, particularly if the drug is an opioid. Furthermore, many drugs are unstable in the HCl salt form and are synthesized as either maleate, citrate or in the acetate form. Electrodes made with other consumable metal would have similar challenges about staining in a similar way.

For the electrotransport of cationic drugs, what is needed is an anode electrode that is able to undergo oxidation without electrolysis of water, which can generate gas, or resulting in staining the tissue.

### SUMMARY

The present invention relates to anodic electrode for the electrotransport delivery of cationic drugs through a body surface and methods of making and using such electrodes. This invention identifies electrode features and methodologies to obtain anodes for cationic drug delivery in electrotransport applications, which can be done without generating a gas or delivering a competing ion or resulting in metal staining in body tissue. The anode includes a precipitate-forming anion source layer that provides anions to react with metal ions generated from sacrificial metal during electrotransport. The present invention provides anodes, electrotransport systems, methods of making and methods of using such anodes and electrotransport systems. There are a number of potent drugs that are therapeutic in the cationic form for desired efficacy, e.g., narcotics such as fentanyl salts and sufentanil salts. These can be delivered iontophoretically with the anode of the present invention without staining the tissue, e.g., skin.

In one aspect, the present invention provides an electrotransport system for administering an intended cationic drug through a body surface. The system includes an anodic reservoir containing the drug and an anodic electrode for conducting a current to drive the drug in the anodic reservoir in electrotransport. The anodic electrode has a polymeric material (e.g., binder material) with metal immobilized (e.g., embedded) in it. The metal during electrotransport forms metal ions. The polymeric material also includes precipitate-forming anions (i.e., anions that are capable of combining with silver ions to form a precipitate, e.g., an insoluble salt AgCl) that can react with the metal ions to form insoluble precipitates in the polymeric material. For example, the anions can be exchanged out of an anion-exchanger chloride source to precipitate with metal ions such as silver ions. The anodic electrode is disposed on a side of the anodic reservoir distal from the body surface so that when the system is applied to the body surface cations migrate in the direction from the anodic electrode through the reservoir to the body surface (e.g., skin) tissue. The metal embedded in the anode can be metal pieces such as particles or mesh.

In another aspect, the present invention also provides methodology for making anodes and electrotransport systems for delivery of cationic drug. To make the anode, an anion source having precipitate-forming anions is included in an anion source layer. The anion source layer is associated with a sacrificial (consumable) metal, which would generate metal ions during electrotransport. The metal ions and the precipitate-forming anions can react to form an insoluble precipitate. The anode is disposed on a reservoir that contains a cationic drug, e.g., fentanyl HCl, sufentanil citrate, and the like, and is connected to a power source and control circuitry to form an electrotransport system.

In another aspect, the present invention also provides methodology for making anodes and electrotransport systems using water-soluble chloride source excipients. To make the anode, water soluble quats such as SENSOMER® CI-50 is formed in conjunction with consumable metal into a solid film and formed into an electrode. The anode is disposed on a reservoir that contains a cationic drug, e.g., fentanyl HCl, and is connected to a power source and control circuitry to form an electrotransport system.

In another aspect of this invention, the use of anodes has also been shown to be useful to deliver non-HCl form of drug with Ag electrochemistry.

In one aspect, the metal is present as pieces of the metal in the anion source layer. The metal pieces can be in the form of particles, beads, flakes, mesh, foils, coil, etc. As used herein, mesh can be considered pieces because of voids in the mesh and light and other material can pass straight through the voids in a mesh. The anion source can also be present in the anion source layer as pieces, e.g., in particulate form of beads or grains. In this way, the metal and the anion source material are commingled for efficient transfer of ions to facilitate precipitation of the reaction product between the metal ion and the anion. In a preferred example, the metal is silver and the anion is halide, especially chloride.

In another aspect, the precipitate-forming anion source can be present in the anion source layer and the layer with the anion source can be disposed on a sacrificial metal support to form the anode. The anion source can also be present in the anion source layer as pieces, e.g., in particulate form of beads or grains. In this way, the metal is not commingled with the anion source material, but is rather upstream (in terms of cation travel path) during electrotransport.

In one aspect, the present invention also provides a method of using a new composite anode and a method using an electrotransport drug delivery system to a body surface with such a new composite anode. The method involves providing an anode as described above and providing anodic reservoir having a cationic drug, connecting the anode to the reservoir and to electrical circuitry to drive the cationic drug for delivery to the body surface and precipitating out the metal ions as insoluble salt in the anode. The anode is applied and connected to the side of the anodic reservoir distal to the body surface. Preferably the anode is a unit structure in which the materials are permanently fixed and irremovable (i.e., irremovable without physically damaging or destroying the electrode), except allowing for ions to pass and liquid can penetrate to allow ion movement.

The present invention provides the advantage that metal staining of body tissue due to metal ions migrating to the tissue in electrotransport is prevented or substantially reduced so that no noticeable staining in tissue (e.g., skin) is observed after the period of electrotransport. The metal ions (formed from the sacrificial metal) are precipitated out as metal salt precipitates in the electrode, more specifically in the anion source layer. In the past, excess amount of cationic drug that contains chloride was needed to minimize the amount of silver staining on the skin, see, e.g., U.S. Pat. No. 6,881,208. With the present invention, because the metal ions (e.g., silver ions) are efficiently precipitated out as metal (e.g., silver) salt in the anodic electrode by precipitate-forming anions in the anode, less drug loading is needed than in the past. Further, with the presence of precipitate-forming anions in the anode, even drugs without the same anion or chloride ions can be used in the cationic drug reservoir. In the embodiment in which the anion source and the metal are commingled in the anode, close proximity between the anions and the metal ions generated in electrotransport allows efficient precipitation reaction to remove the metal ions to prevent them from migrating to the body tissue, or even into the cationic drug reservoir.

The use of a film or layer of firm, tough material containing anion source provides an advantage that the anode is sturdy and can be handled relatively conveniently without risk of damaging compressible material such as a gel. This facilitates ease of use of the electrode and the resulting device. Cationic drugs can be effectively delivered without metal staining. For example, at least 80-100 microgram/cm² hr (μg/cm² hr) of fentanyl base equivalent can be delivered using a current of at 100 microA/cm²(i.e., mcA/cm² or μA/cm²); about 100 μg/cm² hr can also be delivered at 100 μA/cm² without observable silver staining. Cationic drugs can be effectively delivered without metal staining. For example, at least 100 μg/cm² hr (i.e., μg/(cm² hr)) of fentanyl base equivalent can be delivered using a current of at 100 μA/cm² without observable silver staining. Using appropriate composite anodes of this invention, no silver staining was observed up to 10 hour, up to 20 hours, even up to a day of delivery at current flow of 100 μA/cm².

### BRIEF DESCRIPTION OF THE FIGURES

The present invention is illustrated by way of examples in embodiments and not limitation in the figures of the accompanying drawings in which like references indicate similar elements. The figures are not shown to scale unless indicated otherwise.

FIG. 1 illustrates a schematic, sectional view of an embodiment of an electrotransport system of this invention.

FIG. 2 illustrates a schematic, sectional view of an embodiment of an electrode/reservoir portion of this invention.

FIG. 3 illustrates a schematic, sectional view of an anion source layer placed on a drug reservoir of this invention.

FIG. 4A shows a representation of the molecular structure of cross-linked dextran as the support in anion exchange material.

FIG. 4B shows a schematic representation of a quaternary ammonium halide source having an exchangeable halide (e.g., chloride) ion.

FIG. 5 illustrates comparable delivery profile across heat separated human epidermis for the steady state flux and duration using composite anode for two different anode configuration with different supports (namely Ag foil and Ag mesh) compared to a non-composite silver anode.

FIG. 6 shows the amount of silver deposit, i.e., in Ag (determined by ICP-OES) on the skin side gel as a function of duration of electrotransport for A (control, drug loading is taken to be 100% for comparison), B (silver electrode with 60% of drug loading as of the control) and D (Composite anode with 60% drug loading as the control).

FIG. 7 shows the amount of silver deposit, on skin as a function of duration of electrotransport for A (control, drug loading is taken to be 100% for comparison), B (silver electrode with 60% of drug loading as of the control) and D (Composite anode with 60% drug loading as the control).

FIG. 8 shows the flux of fentanyl citrate delivery using the composite (silver mesh) anodic electrode of the present invention.

FIG. 9 shows the comparison of the flux of fentanyl HCl delivery using the composite anodic electrodes with that of control.

FIG. 10 shows the comparative flux during delivery of fentanyl using composite electrodes and a control.

FIG. 11 shows the comparative pH shift after fentanyl delivery using composite electrodes and a control.

FIG. 12 shows the comparative flux during delivery of fentanyl using a composite electrode and a control.

FIG. 13 shows the accumulative flux during delivery of fentanyl using the composite electrode and control of those of FIG. 12.

FIG. 14 shows the comparative pH shift after fentanyl delivery using the composite electrode and control of those of FIG. 12.

### DETAILED DESCRIPTION

The present invention is related to an anode electrode associated in an electrotransport drug delivery system wherein the anode electrode has a polymeric anion (e.g., chloride) source bound to a polymeric material to provide anions (e.g., chloride ions) to react with a metallic ion to form a precipitate during the electrotransport of the drug. Preferably the metal ions are silver ions generated by the oxidation of metallic silver during the electrotransport process. Thus, staining by the metallic ions migrating to body tissue is substantially reduced or prevented, such that it is not observable visually. The system can be applied to deliver drug to a body surface (e.g., transdermally through skin, or across an ocular tissue, such as conjunctiva or sclera). The anode can also be used as counter electrode for the delivery of anionic drug where the cathode will be the donor.

The practice of the present invention will employ, unless otherwise indicated, conventional methods used by those skilled in the art in pharmaceutical product development.

In describing the present invention, the following terminology will be used in accordance with the definitions set out below.

The singular forms “a,” “an” and “the” include plural referents unless the context clearly dictates otherwise. Thus, for example, reference to “a polymer” includes a single polymer as well as a mixture of two or more different polymers.

The term “composite anode” means that the anode has anion source material dispersed in a carrier material. A composite anode can also include metal pieces dispersed therein.

As used herein, unless specified to be otherwise in the content, “distal” refers to a direction pointing away from or being more distant to the body surface, “proximal” refers to a direction pointing to or being nearer to the body surface.

The terms “drug” and “therapeutic agent” mean any therapeutically active substance that is delivered to a living organism to produce a desired, usually beneficial, effect, such as relief of symptoms or discomfort, treatment of disease, or adjustment of physiological functions, e.g., analgesic, regulation of hormone, antimicrobial action, sedatives, etc.

As used herein, the term “immobile” relating to ion source refers to a material that is not driven from the layer the ion source is in electrotransport by the electrical potential present for delivery of the ionic drug. The ion source can be in particulate form, incorporated into particulates, or immobile because of large molecular weight.

The term “pharmaceutically acceptable salt” refers to salts of a drug, e.g., fentanyl, that retain the biological effectiveness and properties, and that are not biologically or otherwise undesirable.

The term “salt” means a compound in which the hydrogen of an acid is replaced by a metal or its equivalent. As used herein, the salt can be in ionized form in solution or in undissociated form (e.g., in solid form). Some salts can also be insoluble in aqueous solutions, e.g., AgCl.

As used herein, the terms “transdermal administration” and “transdermally administering” refer to the delivery of a substance or agent by passage into and through the skin, mucous membrane, the eye, or other surface of the body into the systemic circulation.

### MODES OF CARRYING OUT THE INVENTION

The present invention provides an anode for electrotransport delivery of cationic compounds (e.g., cationic drugs) through a body surface, such as skin or mucosal membrane, e.g., buccal, rectal, behind the eye lid, on the eye such as transconjuctival or transscleral, etc.

Electrotransport devices, such as iontophoretic devices are known in the art, e.g., U.S. Pat. No. 5,503,632, U.S. Pat. No. 6,216,033, US20060089591, can be adapted to incorporate and function with the electrodes of the present invention. The electrotransport drug delivery system typically includes portions having a reservoir associated with either an anodic electrode or a cathodic electrode (“electrode/reservoir portions”). Generally, both anodic and cathodic portions are present. The electrode/reservoir portion is for delivering an ionic drug or counter ions. The electrode/reservoir portion for the drug reservoir typically includes a drug reservoir in layer form that is to be disposed proximate to or on the skin of a user for delivery of drug to the user. The drug reservoir typically includes an ionic or ionizable drug. The typical iontophoretic transdermal device can have an activation switch in the form of a push button switch and a display in the form of a light emitting diode (LED) as well. Electronic circuitry in the device provides a means for controlling current or voltage to deliver the drug via activation of the electrical delivery mechanism. The electronics are housed in a housing and an adhesive typically is present on the housing to attach the device on a body surface, e.g., skin, of a patient such that the device can be worn for many days, e.g., 1 day, 3 days, 7 days, etc. The patents disclosed above related to electrotransport are incorporated by reference in their entireties.

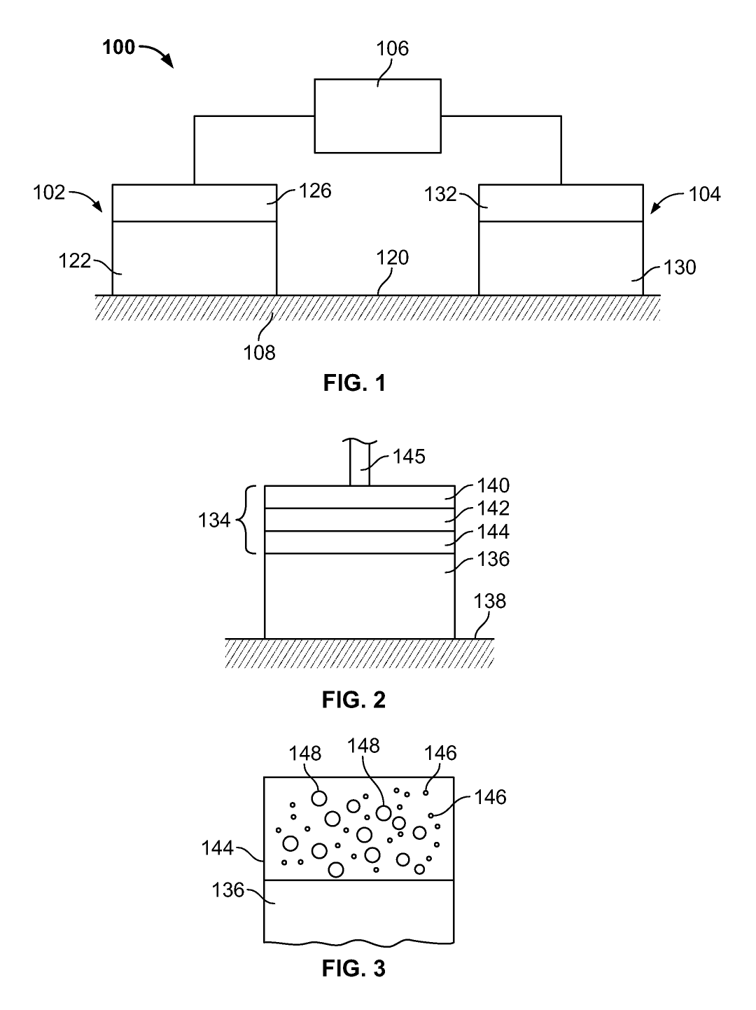

The anode will be illustrated with an anode made with silver and chloride ion source, although other metals and precipitate-forming anions are applicable by one skilled in the art based on the present disclosure. FIG. 1 shows an embodiment of an electrotransport device 100 of the present invention having anode electrode/reservoir assembly 102 and cathode electrode/reservoir assembly 104 connected to and controlled by a controller 106 that provides power source to drive electrical current through the system 100 to the patient's tissue 108 through body surface 120 (e.g., skin surface) of the patient. The anode electrode/reservoir assembly 102 has an anodic reservoir 122 contacting the body surface 120 and an anodic electrode 126 disposed on the anodic reservoir 122 that contains chemical reagents (e.g., donor drug) to be delivered to the patient by electrotransport. The cathode electrode/reservoir assembly 104 has cathodic reservoir 130 contacting the body surface 120 and an electrode 132 disposed on the cathodic reservoir 130. The cathodic electrode is the counter electrode if the anodic reservoir contains cationic drug to be delivered.

The present invention provides anions in the anodic electrode that can form precipitate with the metallic cation generated in the anode during electrotransport. There are a variety of possible electrode electrochemically active component materials and drug anions for sacrificial electrode devices that form insoluble salt precipitates. In general, silver, copper and molybdenum metals form insoluble halide salts (e.g. AgCl, AgI, AgBr, CuCl, CuCl, CuBr, MoCl₃, MoI₂) and therefore are possible sacrificial anode candidates for delivery of cationic drugs. Insoluble precipitates are formed if the solubility product Ksp of the salt is small, typically less than 1.78×10⁻¹⁰ mol²/kg².

FIG. 2 shows an embodiment of an anode electrode/reservoir assembly 134 including anode electrode placed on top of anode reservoir 136 (e.g., a hydrogel, liquid-soaked pad, etc.) disposed on skin surface 138. The anode electrode 134 includes metallic support layer 140 on which side proximal to the body surface is disposed an electrically conductive adhesive 142. On the surface of the electrically conductive adhesive 142 towards the body surface is disposed a polymeric layer 144 of chloride ion source. Electrical connector 145 is connected to metallic support 140 to provide electrical communication to a controller circuitry, e.g., controller 106 (not shown in FIG. 2). It is understood that some of the layers in the embodiment of FIG. 2 can be combined optionally. For example, if desired, the metallic support can be part of the electrical connector. Further, the polymeric chloride ion source can be disposed directly on the metallic support. In this embodiment, preferably there is no additional layer containing a liquid, or a gel or other electrolyte or ion exchanger separately or in combination more distal to the polymeric layer 144 of chloride ion source. Other alternative ways of providing electrical connection to the polymeric layer 144 that contains the chloride source can also be used. For example, the metallic support layer 140 can have varied size and shape and can be made with nonmetallic material such as conductive plastic. One skilled in the art can make other variations in view of the present disclosure.

With the chloride ion source of the present invention in the anode, the anode/reservoir assembly in FIG. 1 and FIG. 2, as well as the cathode/reservoir assembly suitable for an embodiment of FIG. 2, of course, can be part of an electrotransport system with reservoirs, housing, and other features applicable to a body surface for drug delivery use, similar to those shown in U.S. Pat. No. 6,216,033, and the like.

An embodiment of a polymeric chloride ion source layer is generally shown in the schematic illustration of FIG. 3. In FIG. 3, disposed next to the anodic reservoir 136 is the polymeric chloride ion source layer 144, which includes silver pieces (e.g., silver particles) 148 embedded within the layer 144. The polymeric chloride ion source layer 144 also includes embedded therein chloride source particulates 146 on which chloride ions are bound. The silver pieces and chloride source particulates are bound by a binder to form a coating or layer that is solid, preferably not tacky, and generally dry to the touch before applying to a reservoir. When applied to the reservoir, the layer (or coating) allows liquid (e.g., aqueous solution) penetration to carry ions for ionic communication between the layer and the reservoir.

These particulates are a source of the precipitate-forming anions. In the embodiment with chloride ions, the chloride ions are bound to the particulates 146 in an ionic fashion, not covalently, such that the chloride ions can react with, for example, silver ions that migrate there, thereby forming silver chloride, which is insoluble and therefore will participate out in the polymeric chloride ion source layer 144.

It is preferred that adequate sacrificial metal (e.g., silver) is present in the anodic electrode, and the surface area is adequate to allow oxidation at an adequate rate to prevent any significant pH drift during electrotransport in which oxidation occurs in the electrode to generate cations. When oxidizable anodic metal is not adequate or the surface inadequate for forming metal ions, instead of metal being oxidized to form metal cations, water is oxidized in electrolysis, thereby releasing hydronium ions. In electrolysis, gas is also generated. The presence of metal, such as silver in particulate form, such as beads, particles of various shapes, flakes, etc., provides a large surface area for oxidation to take place. Such forms of silver provide more surface area per mass than traditional silver anodes, e.g., a silver foil. Adequate Ag oxidation would reduce pH drift and the release of gas by electrolysis. Also, competing ions (ions of metal such as silver), being precipitated out (such as AgCl) due to the presence of the anion (e.g., chloride) source, are not delivered to the tissue.

In a silver anode electrode, preferably the silver is in a form that is embedded in a polymeric matrix, such as a polymeric chloride ion source layer 144. The silver is preferably in pieces in the form of leaves, beads, grains, particles (nano, micro), foil, wedges, flakes, mesh, and the like. High purity Ag (99.99%) with minimal ionic impurity is preferred. More preferred are particulates such as beads, particles, and flakes that provide a large surface area per volume ratio. For example, small silver particles (such as ranging from 100 nm to 250 microns) are very useful. Nanoparticles of silver (particle size of 100 nm and less) can also be used. Silver flakes of various sizes are commonly available, e.g., with mixed particles sizes of about 1 micron to about 100 microns. Also, silver particles of sizes larger than 100 microns or 250 microns can also be used. It is noted that metal (e.g., silver) in piece form provides a large surface area for oxidation to form metal (e.g., silver) ions and therefore provides higher efficiency for electrical current flow without clogging flow channels easily with the precipitation of less conductive salts (e.g., silver chloride) or other non-conducting material. Thus, silver particles of 250 microns or smaller are preferred. Similarly, other suitable metals described above can be made into anodic electrodes. The size considerations are similar to that of silver.

The anion source for forming precipitate with the metal ions can have a wide variety of anions. Preferably the anion is a halide ion. The preferred anion in the anion source is chloride. In the following, chloride will be used as an illustration for the anion source. It is understood that other halides, such as fluoride, bromide, and iodide can similarly be employed. The precipitate-forming anion source used in the present invention is preferably a macromolecular source of anion (e.g., chloride) so that the anions are bound to the macromolecular material that is insoluble and can be held in a layer without diffusion away easily. For example, the anions are bound ionically to solid phase material such as polymeric beads and particulates distributed in the anode electrode anion source layer. The anion source can be chloride sources where the chloride ions are bound to polymeric material, ion exchange resins with chloride ion as the primary exchangeable ion, or polymeric quaternary ammonium compounds, etc. The polymeric material having bound chloride ions that can react with metal (e.g., silver) ions to form precipitating silver chloride can be anion exchange material. Much of the precipitation will take place in the composite electrode. However, since chloride ions will appear in the gel in which the drug is stored and silver ions can migrate there, precipitates can also form in the gel.

Polymeric material having bound anions can be anion exchangers. Anion exchanger (anion exchange material) can be an organic resin with pendent anionic groups. Examples of anionic selective materials are described in the article “ACRYLIC ION-TRANSFER POLYMERS”, by Ballestrasse et al, published in the Journal of the Electrochemical Society, November 1987, Vol. 134, No. 11, pp. 2745-2749. Example of other anion exchange material would be a copolymer of styrene and divinyl benzene reacted with trimethylamine chloride to provide an anion exchange material (see “Principles of Polymer Systems” by F. Rodriguez, McGraw-Hill Book Co., 1979, pgs 382-390). These articles are incorporated herein by reference in their entirety. Methods for making anion exchange material are known in the art. Typically such methods involve polymerization and cross-linking to produce polymeric material that is insoluble in water. Such ion exchange material can be made into particulates and membranes. Although the anion exchange materials are preferably porous to allow ions to pass through, it is preferred that they do not swelling excessively, since swelling may cause delamination and separation of the anion source later to from the anodic electrode.

For anionic exchange materials of the present invention, strong anionic functionality (such as quaternary ammonium type anion-exchange resin) is desired. Useful anion sources include polymeric amines and preferred are polymeric tertiary and quaternary ammonium compounds on which anions (e.g., chloride ions) are held ionically and from which the anions (e.g., chloride ions) can react with metal ions (e.g., silver ions) to form precipitate (e.g., insoluble AgCl).

Generally, more useful chloride sources include polysaccharide-based materials that can release anions such as halide ions (e.g., chloride ions) to react with metal ions such as silver ions to form precipitates. Such polysaccharide-based polymeric chloride sources have a polysaccharide backbone or a backbone that is derived from polysaccharide. The backbone is therefore a chain containing monosaccharide units, such as glucose, linked by glycosidic bonds. Examples of polysaccharide-based materials that have ionic capacity are SENSOMER® CI-50 from Ondeo Nalco, Naperville, Ill. (which is a cationic starch derivative, i.e., Starch Hydroxypropyltriammonium Chloride) and SEPHADEX™ QAE, a quaternary aminoethyl dextran-based resin cross-linked with epichlorohydrin. SENSOMER® CI-50 is a cationic polysaccharide derived from food grade potato starch that is free of environmental toxic residues. The monosaccharide in starch is glucose. The average molecular weight of SENSOMER® CI-50 is about 2×10⁶ Dalton. It has been reported that no clinically significant responses were seen with SENSOMER® CI-50 material on any of the subjects who participated in a human repeated-insult study. Tests have shown that SENSOMER® CI-50 was neither a skin irritant nor a skin sensitizer. None of the substances in SENSOMER® CI-50 are listed as carcinogens by the International Agency for Research on Cancer (IARC), the National Toxicology Program (NTP) or the American Conference of Governmental Industrial Hygienists (ACGIH). SENSOMER® CI-50 is biocompatible and has been used in hair products (e.g. shampoo, conditioner) and skin-care prodtucts (e.g., cream, lotion), When incorporated into the electrode, the SENSOMER® CI-50 is considered to be immobile because of its large molecular weight. Halide ion such as chloride ions that associate with SENSOMER® CI-50 can react with metal ions such as silver ions to form precipitates. SENSOMER® CI-50 be used in conjunction with sacrificial metal (e.g., silver) particles to form a film or particles, or can be embedded in porous particles and incorporated (or bound) into the electrode by using binders.

SEPHADEX™ ion exchange resins are dextran-based and therefore the monosaccharide in its backbone is also glucose. SEPHADEX™ ion exchange resins are available from Sigma-Aldrich commercially (e.g., in 2007 A.D.). A more preferred material is SEPHADEX™ QAE A-25, which is a SEPHADEX™ strong ion exchanger. It is contemplated that other biocompatible anion exchange resins can also be used. Particulate anion exchange material typically absorbs aqueous liquid and swells to release the exchangeable ion, thus allowing precipitation reaction. We have found that excessive water uptake by the electrode via swelling is not preferable since it may lead to the anion source layer coming off (separating) from the support at one or more spots in the anodic electrode. Such separation of layer from the support can have the appearance of wrinkling or fluffiness. Also, inadequate binder would lead to separation of the composite coating layer from the support. Further, without an adequate amount of binder, the composition may not result in a smooth coating. Preferably, the swelling by absorption of liquid upon contact with the reservoir is 2.5 gram per gram of anion exchange resin, or less. Typically for SEPHADEX™ QAE A-25, the swelling is about 2.5 gram of water per gram of dry powder and therefore is a preferred anion exchanger. The swelling in weight ratio can be determined by applying an anodic electrode of the present invention onto a hydrogel (e.g., PVOH hydrogel), seal the two together in a vapor-tight pouch and let them equilibrate for an adequate period (such as 15 hours) in a constant temperature (e.g., room temperature), and find out the weight loss by the hydrogel after the equilibration period. One can determine the water loss of the hydrogel by weighing the hydrogel before attaching the anodic electrode and weighing the hydrogel after separating from the anodic electrode after the equilibration period. Knowing the amount of anion exchange resin in the anodic electrode, the water absorption in weight ratio (related to swelling) by the anion exchange resin and by anion source layer in the electrode can be calculated.

Because water absorption by the electrode would consequently reduce the moisture content of the reservoir during electrotransport, it is preferred that the water absorption by the ion exchanger be no more than about 300 wt %, preferably about 250 wt % or less. However, water absorption also functions to facilitate ion movement within the electrode. Thus, it is preferred that in the electrode, referring to the material with the anion exchanger particulates more or less homogenously, uniformly, or evenly mixed in before water absorption, have a water uptake capacity of about 10 wt % to 300 wt %, preferably about 20 wt % to 250 wt %. Generally, anodic electrodes are applied to a drug reservoir to cover 80% to 100% of the surface of the drug reservoir facing the electrode. Although it is desirable that the composite electrode absorbs some water to allow ion movement, the composite electrode is designed that typically it does not absorb a significant amount of water from the drug reservoir. Water absorption tests were done by placing a 0.5 inch (1.27 cm) diameter, 1/16 (1.6 mm) inch thick polyvinyl alcohol hydrogel typically used iontophoretic delivery (similar to what is used in the IONSYS™ system) into a well of the same size in a substrate of the same thickness. An occlusive release liner was laid on top covering the hydrogel and the composite electrode with the about the same surface area as the hydrogel was placed under the hydrogel in contact therewith. An occlusive backing layer larger in area than the composite electrode was placed under the composite electrode and then the whole system was placed in a water vapor tight pouch. Systems were weighed at different time intervals to determine the amount of water transferred from the hydrogel into the composite electrode. In this way the steady state water uptake by the composite electrode was determined.

Water soluble halide source such as SENSOMER® CI-50 material can be used for forming the anode in conjunction with consumable metal (e.g., silver) pieces, such as particulates (flakes, particles, beads, etc.). SENSOMER® CI-50 material usually is supplied as a 31-33 wt % dry basis aqueous solution at pH about 3.5-4.5 at room temperature. The soluble halide source can be dispersed with the metal pieces in a solution of the binder dissolved in a solvent. The metal pieces (e.g., Ag) and the halide source can be mixed well in the binder solution and then the solvent is removed from the mixture to render a film with the halide source and the metal (e.g., Ag) pieces embedded in the binder matrix. Water that is in the SENSOMER® material is also evaporated in the drying process. The film can further be divided to form pieces resembling particles. The mixture with the binder solution and metal pieces can further be make directly into particulates and dried. Particle making processes are known to those skilled in the art.

For the anion exchange material that comes in a suspension of solid particles in an aqueous liquid, the particles are removed from the liquid and mixed with a polymeric binder and cast on a surface to form a layer. It is to be understood that the above ion exchange materials may be used in other halide forms.

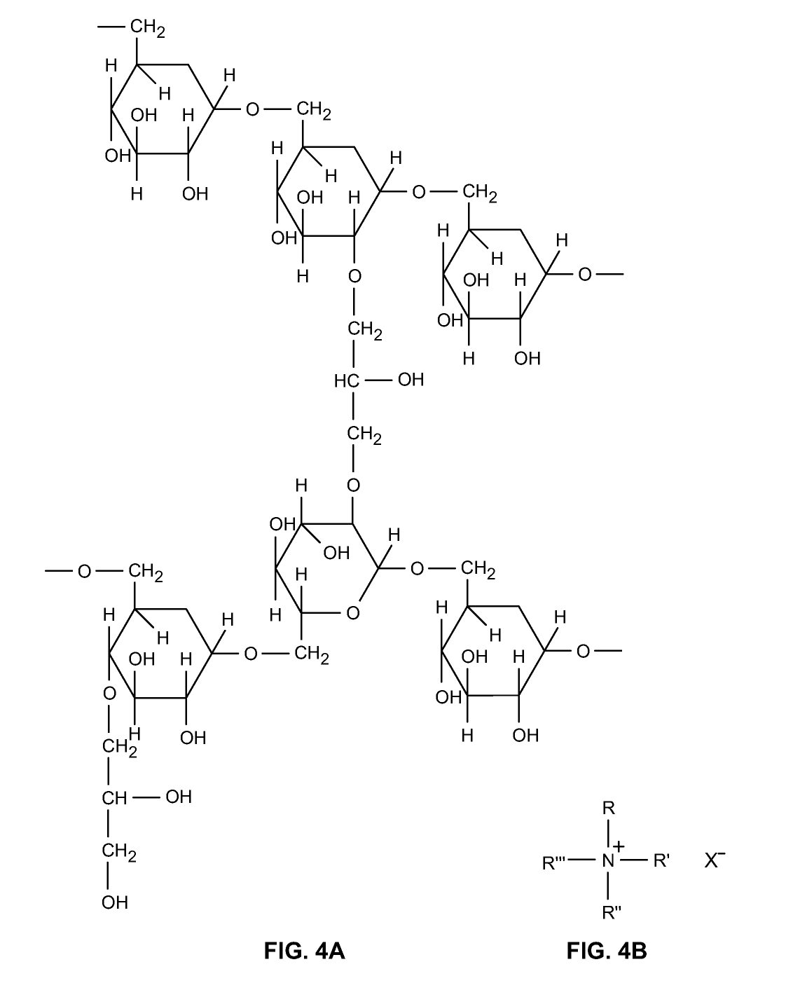

FIG. 4A to FIG. 4B show examples of polymeric anion sources and how they ionically hold on to anions (e.g., chloride ions), which are capable of reacting with metallic ions to form a precipitate. FIG. 4A shows the molecular structure of dextran showing the cross-link between two dextran chain units. The cross-linked dextran scaffold can be modified to include functional groups to render anionic or cationic exchanging capabilities. SEPHADEX™ ion exchange resin is an example of a dextran-based resin. SEPHADEX™ QAE A-25 and SEPHADEX™ QAE A-50 have quaternary ammonium functionality on a cross-linked dextran supporting carrier structure. SEPHADEX™ is a dry bead material formed by cross-linking dextran with epichlorohydrin. The SEPHADEX™ QAE A-25 and A-50 are anionic exchangers. Such beads will swell when placed in contact with aqueous solution. The A-25 has more cross-linked than the A-50 and tends to swell less. The SEPHADEX™ DEAE anion exchanger has weak anion exchange functionality and remains charged at pH of 2-9. DEAE resins also have A-25 and A-50 varieties. Both QAE and DEAE resins have bead size of about 40 microns to 120 microns. The SEPHADEX™ QAE anion exchanger is a strong anion exchanger and has diethyl-(2-hydroxypropyl)aminoethyl functionalities and is preferred in the present invention. SEPHADEX™ DEAE is 2-(diethylamino) ethyl-SEPHADEX™, i.e., diethylaminoethyl derivative of a cross-linked dextran. Strong anion exchangers are resins that remain charged and have high capacity at working pH of 2-12. For weak anion exchangers, not all the anion exchange functionalities are completely ionized at about pH 2-9. Generally, strong anion exchangers are derived from strong bases and weak anion exchangers are derived from weak bases. Tertiary or quaternary ammonium resin can be useful for anion exchange. Quaternary ammonium resins are especially useful for making strong anion exchangers. Strong anion exchangers, e.g., quaternary ammonium resins, are those anion exchangers that are permanently charged under working pH of 2-10, as understood by those skilled in the art. The A-25 has more cross-linking than the A-50 and tends to swell less. The pore size of A-25 has about 30,000 Da exclusion limit and the A-50 has about 200,000 Da exclusion limit. SEPHADEX™ ion exchange resins are available from Sigma-Aldrich in dry powder form commercially (e.g., in 2007 A.D.). A more preferred material is SEPHADEX™ QAE A-25. Preferably the ionic capacity of the dextran based ion exchange has ionic capacity of 2.5 to 4 mmol/g dry basis, more preferably 2.5-3.5 mmol/g dry basis.

FIG. 4B shows a schematic representation of a quaternary ammonium halide source (having a halide X⁻ associated with the quaternary ammonium ion), which halide can react with the metal ion to precipitate. It is understood that although the SEPHADEX™ anion exchange resin is used in the Examples herein, other anion exchange resin can also be used, especially other strong anion exchangers, since halide ions can be exchanged in similar manners in different anion exchange resins and particulate ion exchange resin can be formulated into composite coating on a composite electrode based on the teaching of the present disclosure. Many strong and weak anion exchanger resins are available commercially as known to those skilled in the art.

The layer of polymeric precipitate-forming anion source can include sacrificial metal that will generate metal ions during electrotransport. The layer, for example, can be formed by including the silver pieces and chloride ion source material (e.g., anion exchanger particles or beads) in a polymeric matrix (carrier material). For example, silver pieces (e.g., silver particulates) and anion exchanger beads in chloride form can be bound by a polymeric binder. For example, polyvinylidene difluoride (PVDF), a thermoplastic fluoropolymer, is a preferred binder for binding the silver pieces and anion exchanger pieces (e.g. particulates). The binder is used for holding, binding the metal, e.g., Ag, and ion exchangers to a substrate for forming a film, coat, or layer in the electrode. Thus, conventional binders that have such a function can be used. Other binders suitable for use include polyisobutylene, acrylics such as those formed from acrylate monomers such as hydroxylethyl acrylate, ethyl hexyl acrylate, butyl acrylate, methyl acrylate; PHMA poly(hexyl methacrylate); PEHMA poly(2-ethylhexyl methacrylate); PLMA poly(lauryl methacrylate) HPMA; and poly (hexamethylene adipate) PHA; styrene-butadiene rubber SBR, polyurethane, etc. Polyurethan is a useful binder. A urethane linkage can be produced by reacting an isocyanate group (—N═C=O) with a hydroxy group. Polyurethane can be produced by simple addition polymerization reaction. It is easy to cure and is soluble in acetone and alcohol (low boiling solvents). Polyurethanes are commercially available. Among the various kinds of binders, fluoropolymers (such as PVDF) are preferred because of their lower water absorption property. Other fluoropolymers such as polytetrafluoroethylene PTFE can be used. A suitable solvent for dissolving the binder (e.g., PVDF), for forming a mixture with the silver pieces and the anion exchanger is N-methyl pyrrolidone (NMP). PVDF is also preferred because of its favorable properties during gel dispensing, in that the electrodes do not curl or wrinkle as the electrode material absorbs liquid from the gel. Other than NMP, we have found that another very useful solvent for PVDF (or a material that is primarily PVDF) is propylene carbonate. NMP or propylene carbonate are preferred solvent for PVDF. Using either NMP or propylene carbonate, it was possible to make composite electrodes that is pH stable for one day of iontophoretic flux of a drug such as fentanyl HCl. Other than NMP and propylene carbonate, other suitable solvents for PVDF or a material that is primarily PVDF for making a composite coating that will not drift in pH to a significant degree are ethyl acetate and toluene. For most other common solvents, it was found that the dispersion mixture having PVDF will have different flow properties and will not result in a good coating). Other usable solvents for other binders include hexane, isopropyl alcohol IPA, acetone, ethyl acetate, ethanol, methyl ethyl ketone, heptane and the likes. Generally, an amount of solvent is used adequate for dissolving the selected binder and rendering the solvent, binder, chloride source material suitable for forming a layer by a layer forming process, such as casting and drying. Other solvents known in the art that can dissolve the binders can also be used such as propylene carbonate, ethyl acetate etc. Solvent removal processes commonly practiced in the field, such as by heat, air circulating, under suction or vacuum to create reduced pressure to facilitate solvent evaporation, can be used for drying the cast material. Addition of high MW plasticizers known in the art such as PEG (1-5% loading and MW 10000-50,000 or above) that does not leach out of the electrode during iontophoresis can also be used in the electrode formulation. Preferably, after solvent removal the composition solidifies into a coat, the coat is not tacky, and is dry to the touch for better handling and operation. The coating when dry is solid, preferably firm with a good surface finish that is uniform. The coat, when in use and in contact with a reservoir, will not become soft, gel-like or easily pealed off. Thus, the binder is different from gel-forming hydrophilic or water-soluble material such as polyvinyl alcohol or hydroxyethylcellulose that would absorb a large amount of water to form a gel. For comparison, a gel is a material that is jelly like, although able to maintain a shape under normal gravity, is soft to the touch and gives easily under light finger pressure.

The binder functions in providing a polymeric solid structure holding the particles together. The binder is preferably capable of being made into a liquid form, either by thermoplastic melting or preferably by dissolving using a solvent. After a composition of the binder with the particles is cast to form a composition layer, the composition layer will solidify either by cooling or through the vaporization of the solvent. In this way, a solid electrode layer containing the particles bound by the polymeric binder is formed. For binders, such as PVDF and PIB, the optimum dry binder weight % was found to be in the 13-16% range. For PVDF, concentrations higher than 16 wt % may result in higher resistance. At concentrations much lower than 13-14 wt % (e.g., lower than 11 wt %), the composite slurry becomes too fluid and may not have the property suitable to be cast. A viscous finish is required for good castability. We have discovered that using a slow solvent removal method helps to prevent cracking of the film. PVDF of MW of above 300,000 Da is useful. PVDF is commercially available (e.g., SOLEF 6020, Solvay SA, Belgium, and Sigma Aldrich), e.g., as Product No. 182702 from Sigma Aldrich with molecular weight 534,000, about 0.5 million MW. We have found that other hydrophobic fluoropolymers are useful, similar to PVDF, e.g., tetrafluoroethylene. A composite slurry with PVDF can be cast on a electrically conductive adhesive tape (E-CAT) and there need not be a silver foil in the anode electrode. Such a composite electrode without silver foil can function well in delivering cationic drugs such as fentanyl, without allowing moisture to migrate to the back of the electrode to the electronics. However, if desired, silver foil can also be included more distal to the polymeric composite layer having the anion source, e.g., more distal from the skin and attached to the E-CAT.

Another preferred binder is polyisobutylene (PIB). Typically PIB binders are a mixture of high molecular weight PIB (HMW PIB) and low molecular PIB (LMW PIB). PIB has excellent binding property and is suitable for use as binder in the present invention. PIB mixtures are described in the art, e.g., U.S. Pat. No. 5,508,038. The molecular weight of the HMW PIB will usually be in the range of about 700,000 to 2,000,000 Da, whereas that of the LMW PIB will typically range from about 1,000 to about 60,000, preferably from 35,000 to 50,000. The term, “moderate molecular weight polyisobutylene” (MMW PIB) refers to a polyisobutylene composition having an average molecular weight in the range of higher than about 60,000 to smaller than about 700,000. The molecular weights referred to herein are weight average molecular weight. The weight ratio of HMW PIB to LMW PIB in the useful adhesive will normally range between 2:1 to 1:4, preferably 3:2 to 2:3, more preferably about 1:1.

For PIB binder, optimum loading of high MW to low MW PIB is important to obtain electrode materials that are non tacky when processed with heptane as the solvent. For example, an optimum ratio of 1:1 (VISTANEX LM-MS or OPPANOL B12: VISTANEX MM L-100 or OPPANOL B100) has been found to be optimum to obtain electrodes that do not have surface tackiness. The nominal molecule weights of VISTANEX LM-MS, OPPANOL B12, VISTANEX MM L-100, and OPPANOL B 100 are 35 k, 60K, 1.2M and 1.1 M respectively. For nontacky electrode surfaces, the dry weight percent of PIB in the final films was found to be close to 13 wt % with a range of 12 wt %-14 wt % being the optimum. PIB should typically ranges between 10 wt %-16 wt %. The ratio of HMW and LMW affects the property of the composite. PIB composite electrode with PIB concentrations below 13 wt % may result in tackiness while electrodes with PIB concentrations in the 14-16 wt % were found to have very high resistivity when the ratio of high to low MW PIB was 1:4. The electrode films with PIB compositions farther away from the optimum value of 13 wt % dry weight were found to require high voltage for operations under iontophoretic conditions and also caused pH shift of the drug formulation during iontophoresis.

Further, it was found that the thickness of the coating had an effect on the pH and in vitro flux performance of the PIB composite electrode. Typically the thickness should be above about 3.5 mils (0.087 mm), more preferably above about 6 mils, more preferably about 6-10 mils (0.15 mm-0.25 mm). A thickness of less than about 3 mils (0.075 mm) coating showed both pH shift and poor flux. Evaluation of the thickness of the coating layer revealed that for a current density of about 100 μA/cm², a coat thickness of at least about 6 mils (0.15 mm) is useful to maintain the pH and steady state flux. However, it is contemplated that one skilled in the art, based on the present disclosure, will be able to adjust the thickness, ratio of HMW to LMW PIB to arrive at an electrode that is somewhat different from the optimal conditions described above using different molecular weight HMW PIB and LMW PIB.

One way of making the anode, e.g., anion source (chloride-containing) anodic electrode, is by mixing, e.g., silver particles and the chloride ion source material (e.g., anion exchanger beads) in a binder/solvent mixture followed by solution-casting to form a layer. For example, casting of the mixture can be done on an electrically conductive adhesive tape (E-CAT). The E-CAT containing the composite anode mixture then can be dried to remove the solvent. Drying can be done, e.g., by placing the cast material in a heated air furnace at 100° C. for 1 hr.

An alternative way to make the anode electrode is to form a layer of the polymeric anion source (e.g., one that contains silver particles) and laminate it to an electrically conducting tape (E-CAT) to form an anion source laminate (as done with the PVDF composite material). With the E-CAT present, the anion source laminate can be affixed to an electrical connector or conductor to have electrical communication to the power source and control circuit.

In general, the process of making an anode involves these steps: Dissolve the binder in a suitable solvent (e.g., PVDF in NMP) completely. Mix silver particles or flakes and anion exchange material. Combine these together and mix the composition well in a mixing equipment till a grayish slurry is obtained; the viscosity of the slurry is expected to be around 3-6 poise at 50-100 RPM using a Brookfield CAP 2000 viscometer. Cast the slurry on an E-CAT or a release liner and dry off the solvent. For forming a laminate anode, cast the slurry formed above on a release liner instead of E-CAT and then laminating the cast layer with E-CAT. Obviously, when other binders, metal pieces and other ion exchangers are used, they can be adapted for the above process to make an electrode in a similar manner.

Other processing methods include screen printing and lamination by standard methods for people known in the art.

The presence of precipitate-forming anion (e.g., chloride) source in the anodic electrode reduces the extent of metallic staining (e.g., silver staining if the electrode contains silver) on body tissue. Generally, the amount of precipitate-forming anion (e.g., chloride) loading in the anodic electrode is such that substantially all the metal ions (e.g., silver ions) generated by the metal (e.g., silver) during the electrotransport process can be precipitated out so that any metal (e.g., silver) staining of the body surface of the patient is eliminated or reduced to the extent that it is unnoticeable by visual observation. It is understood that, however, even if a little reactable precipitate-forming anion (e.g., chloride) present will help to reduce staining due to the metal (silver in the case of a silver-containing electrode) migration. Preferably the anion (chloride ions) loading is such that at least enough anions (e.g., chloride ions) are present in the chloride ions source stoichiometrically equivalent to the metal (e.g., silver ions) that will be generated by the device during the intended period of electrotransport. Since a device is designed to function for a predetermined period of time for a predetermined amount of electrical energy to pass through to deliver a predetermined amount of cationic drug, the stoichiometric equivalent of the metal ions (e.g., silver ions) to be generated can be known and the equivalent amount or more of the anion (e.g., chloride ions) can be included in the anion source before the device is used.

A sufficient amount of solid or polymeric material to which the precipitate-forming anions are bound (associated) is present for the loading of anions (e.g., chloride ions). For example, at least an adequate amount of anion exchange resin is present for the chloride ions to be held to combine with the stoichiometric equivalent of the silver ions that will be generated in the electrotransport. Knowing the type of anion exchange material being used and the amount of chloride ion loading available (exchange capacity), the right amount of the chloride form of the anion exchange material can be included in the anodic electrode chloride source layer. Knowing the type of anion exchange material to use, one skilled in the art can readily calculate, as well as experimentally determine the amount of the ion exchange material to use in the anodic electrode chloride ion source layer. Obviously, anions other than chloride, such as other halides, can similarly be employed by those skilled in the art based on the present disclosure.

Knowing the amount of the cationic drug that is to be delivered, one skilled in the art can calculate the amount of metal (e.g. silver) ions that will be generated and the amount of sacrificial metal to be included in the anode using Faraday's law, and therefore the amount of metal to include. Preferably the metal particles (e.g. Ag particles or flakes) have particle size of about 100 nm to 50 μm and preferably about 0.5 to 10 m. For example, Sigma-Aldrich 10 micron silver flakes CAS Number 7440-22-4 Product Number can be used. This silver material has a maximum particle size of 10 microns, and 0.8 micron average particle size.

Generally the binder material is present in an amount to securely bind the metal (e.g., silver) pieces and the anion source particulates to allow current flow during electrotransport. Generally, when a binder, e.g., PVDF is used, the ratio of binder (e.g., PVDF) to anion exchanger (in chloride form) dry weight is in the range of about 1:1 to 1:9, preferably about 1:1. The ratio of silver to anion exchanger is about 6:1 to 1:10, preferably 5:1 to 6:1. For example, when SEPHADEX™ anion exchange resin is used, a useful ratio of Ag:SEPHADEX™ resin is 1:9. This will result in a chloride ion source layer that allows silver ions and chloride ions to come together therein to react. Preferably the chloride ion source particulates (e.g., anion exchanger beads) have average diameter in the range of about 40 microns to about 120 microns.

In compositions for forming the electrode via a solvent mixing and drying process involving a binder, preferably the binder and solvent constitute about 30 wt % to 70 wt %, more preferably about 40 wt % to 60 wt %, even more preferably 45 wt % to 55 wt % of the composition. Generally there must be enough binder to form the layer of polymeric material with embedded metal pieces and anion exchanger particulates. There must also be enough solvent for dissolving the binder and for accommodating the particulates and pieces of the metal and anion exchanger in a slurry applicable for forming an electrode. Generally, the binder to solvent ratio is preferably about 1:7 to 1:20, preferably about 1:10. The binder can be dissolved in the solvent and the solution be used for mixing the metal pieces and anion exchanger particulates. Alternatively, the solid materials including the metal pieces, anion exchanger particulates, and binder can all be mixed into the solvent to form the composition. On dry solids basis (not including solvent), the binder in the particle-composite material is about 4 wt % to 30 wt %, preferably about 6 wt % to 20 wt %%, even more preferably 8 wt % to 15 wt %.

In the embodiments in which a continuous piece or a few (e.g., less than 5) pieces of metal (e.g., mesh, or foil) is used in the electrode, less metal pieces of small dimensions, e.g., particles with less than 1 mm across in average particle size, will be needed. In such embodiments, the continuous pieces such as mesh and foil provides much of the surface for generation of metal ions. For example, when a metal mesh or foil having the overall size covering about the gel surface facing the electrode, the corresponding metal pieces (e.g., flakes, beads, powder) to anion exchanger particulates by weight is about 6:1 to 1:10, preferably 5:1 to 1:10, more preferably 2:1 to 1:1. Of course, a relatively high silver to anion exchanger ratio (e.g., 6:1 to 4:1, or 6:1 to 5:1) can be used if cost of silver is not a concern. To make a composition having a binder and solvent that can be later dried to form the particle-composite material, preferably the silver concentration in the slurry is less than about 60 wt %, preferably about 20 wt % to 60 wt %, more preferably about 20-50 wt %, more preferably less than about 40 wt %, even more preferably 30-40 wt %. As used herein, a particle-composite material is the material formed with a polymer having substantially even distribution of metal pieces (e.g., particulates such as flakes, beads, power particles, etc.) and anion exchanger particulates (e.g., beads, particle bits, etc.) therein, preferably in dry form. Thus, in anodic electrode having metal mesh or foil, the mesh or foil will be disposed next to and contacting a layer of particle-composite. In such a slurry composition for making particle-composite, preferably the metal and the anion exchanger account for about 40 wt % to 60 wt %. The anion exchanger in the slurry is about 5-25 wt %, preferably 6-18 wt %, more preferably less than 10 wt %, e.g., 6-10 wt %.

In an anodic electrode layer, i.e., the polymeric layer that contains the metal pieces and anion source (e.g., ion exchanger) on solids basis (i.e., dry basis) comparing without solvent or other vaporizable material, the metal pieces are about 30 wt % to 80 wt %, preferably about 60 wt % to 75 wt %, even more preferably 70 wt % to 75 wt %, even more preferably about 73 wt %-74 wt %. The anionic exchanger is about 5 wt % or more, preferably 5 wt % to 20 wt %, preferably 10 wt % or more, preferably about 10 wt % to 15 wt %.

In embodiments in which there is no continuous pieces of metal (such as mesh or foil) in the electrode (i.e., the metal source is all in the particle-composite material), more particulate metal is needed than in the electrodes with mesh or foil to provide the surface and material for forming the metal ions. In such embodiments, the ratio of metal pieces (e.g., flakes, beads, powder) to anion exchanger particulates is about 10:1 to 2:1, preferably 7:1 to 5:1, more preferably about 6:1 to 5:1.

Optionally, plasticizers (e.g., PEG poly ethylene glycol) can be added during processing to improve the flexibility of the electrode so that the resultant electrode will not break or crack during the making process (e.g., putting on rolls) and while putting at various contours on the body surface. Other plasticizers and material that modifies the modulus known in the art can also be used. Common plasticizers known in the art include such as, e.g. adipic acid esters, phosphoric acid esters, phthalic acid esters, polyesters, fatty acid esters, citric acid esters or epoxide plasticizers. Materials that can affect flexibility of the anode anion-source layer also include hydrogenated oils, hydrocarbon resins, etc. The anode when finished has a plastic appearance and feel and is preferably firm and uncompressible to the touch.

An alternative embodiment of an anode of the present invention is one in which an anion exchanger, instead of being particulates bound in a polymer, is incorporated into the polymeric material as part of the polymeric material. Methods for making ion exchange resins and films are known in the art. See, e.g., pages 52-55 of “A First course in ion permeable membranes”, T. A. Davis, J. D. Genders, D. Pletcher, The electrochemical consultancy, England, 1997, which is incorporated by reference herein. In this case, the metal pieces (e.g., silver particulates) are mixed into the liquid monomers before polymerization. As the monomers are polymerized and solidify, the metal pieces (e.g., silver particulates) are affixed in place and embedded in the polymeric material. In this way, preferably, the metal pieces (e.g., silver particulates) are dispersed among the ion exchange functionality groups evenly. The concentration of metal, e.g., silver) in the anode on dry basis can be similar to the above-described concentrations for anodes made by slurry casting using a solvent and binder. For example, a composition having poly(vinylchloride), styrene, divinylbenzene, 4-ethylbenzene, 2-methyl-5-vinylpyridine, benzoyl peroxide, and dioctyl phthalate are mixed into a paste. Silver flakes are then added and mixed evenly. The composition is heated at about 350-390° K to polymerize and form a layer. The anionic exchange functionalities are then introduced by reacting the layer with suitable agents. For example, the polymerized layer can be soaked in 50:50 chloromethyl methyl ether” carbon tetrachloride containing 5 vol % SnCl₄ at 283° K to introduce chloromethyl groups and then quarternizing by treatment with a trimethylamine solution. Alternatively, to introduce the chloromethyl group, chloromethyl styrene can be included as one of the monomers in the polymerization reaction, before the quaternization. An alternative method of making anion exchange layers involves including vinylpyridine as one of the monomers and following up the polymerization with quaternization using a solution of methyl iodide in petroleum ether. In such cases in which monomers are polymerized and/or cross-linked to for a solid material, the polymeric material can also be considered a binder for binding the metal pieces within the polymeric material in the layer.

The reservoir of the electrotransport delivery devices typically contains a gel matrix (although other non-gel reservoirs, such as spongy or fibrous pads holding liquid, and membrane confined reservoirs, can also be used instead), with the drug solution uniformly dispersed in at least one of the reservoirs. Gel reservoirs are described, e.g., in U.S. Pat. Nos. 6,039,977 and 6,181,963, which are incorporated by reference herein in their entireties. Suitable polymers for the gel matrix can contain essentially any nonionic synthetic and/or naturally occurring polymeric materials. A polar nature is preferred when the active agent is polar and/or capable of ionization, so as to enhance agent solubility. Optionally, the gel matrix can be water swellable. Examples of suitable synthetic polymers include, but are not limited to, poly(acrylamide), poly(2-hydroxyethyl acrylate), poly(2-hydroxypropyl acrylate), poly(N-vinyl-2-pyrrolidone), poly(n-methylol acrylamide), poly(diacetone acrylamide), poly(2-hydroxylethyl methacrylate), poly(vinyl alcohol) and poly(allyl alcohol). Hydroxyl functional condensation polymers (i.e., polyesters, polycarbonates, polyurethanes) are also examples of suitable polar synthetic polymers. Polar naturally occurring polymers (or derivatives thereof) suitable for use as the gel matrix are exemplified by cellulose ethers, methyl cellulose ethers, cellulose and hydroxylated cellulose, methyl cellulose and hydroxylated methyl cellulose, gums such as guar, locust, karaya, xanthan, gelatin, and derivatives thereof. Ionic polymers can also be used for the matrix provided that the available counterions are either drug ions or other ions that are oppositely charged relative to the active agent. It is to be understood that the application of the anodes and devices of the present invention is not limited by the reservoir carrier material so long as the reservoir can function to dissociate drug salts and allow ions to migrate therein. For example, a reservoir that has a semiporous membrane containing a liquid, or a porous pad holding liquid are also applicable for use with an anodic electrode of the present invention.

In certain embodiments of the invention, the reservoir of the electrotransport delivery system comprises a polyvinyl alcohol hydrogel, as described, for example, in U.S. Pat. No. 6,039,977. Polyvinyl alcohol hydrogels can be prepared, for example, as described in U.S. Pat. No. 6,039,977. The weight percentage of the polyvinyl alcohol used to prepare gel matrices for the reservoirs of the electrotransport delivery devices, in certain embodiments of the methods of the invention, is about 10% to about 30%, preferably about 15% to about 25%, and more preferably about 19%. Preferably, for ease of processing and application, the gel matrix has a viscosity of from about 1,000 to about 200,000 poise, preferably from about 5,000 to about 50,000 poise.

Because of the anion source in the anodic electrode precipitates out metal ions generated in the anode, the electrode is applicable to cationic drug delivery of a wide variety of drugs as long the drug can have cationic function and can be included in a reservoir to be delivered iontophoretically. Drugs having cations that can be delivered include analgesics, antitumor drugs, antibiotics, histamines, and hormones. Examples of cationic drugs that can be delivered include, e.g., amiloride, digoxin, morphine, procainamide, quinidine, quinine, ranitidine, triamterene, trimethoprim, or vancomycin, procain, lidocaine, dibucaine, morphine, steroids and their salts. For example, hydrochloride salts of vancomycin, procain, lidocaine, dibucaine, and morphine, and acetate salt of medtroxyprogesterone are cationic drugs that can be delivered. Examples of analgesic drug that can be delivered include narcotic analgesic agent and is preferably selected from the group consisting of fentanyl and functional and structural analogs or related molecules such as remifentanil, sufentanil, alfentanil, lofentanil, carfentanil, trefentanil as well as simple fentanyl derivatives such as alpha-methyl fentanyl, 3-methyl fentanyl and 4-methyl fentanyl, and other compounds presenting narcotic analgesic activity such as alphaprodine, anileridine, benzylmorphine, beta-promedol, bezitramide, buprenorphine, butorphanol, clonitazene, codeine, desomorphine, dextromoramide, dezocine, diampromide, dihydrocodeine, dihydrocodeinone enol acetate, dihydromorphine, dimenoxadol, dimeheptanol, dimethylthiambutene, dioxaphetyl butyrate, dipipanone, eptazocine, ethylmethylthiambutene, ethylmorphine, etonitazene, etorphine, hydrocodone, hydromorphone, hydroxypethidine, isomethadone, ketobemidone, levorphanol, meperidine, meptazinol, metazocine, methadone, methadyl acetate, metopon, morphine, heroin, myrophine, nalbuphine, nicomorphine, norlevorphanol, normorphine, norpipanone, oxycodone, oxymorphone, pentazocine, phenadoxone, phenazocine, phenoperidine, piminodine, piritramide, proheptazine, promedol, properidine, propiram, propoxyphene, and tilidine. For more effective delivery by electrotransport such as iontophoresis, salts of such analgesic agents are preferably included in the drug reservoir. Suitable salts of cationic drugs, such as narcotic analgesic agents, include, without limitation, acetate, propionate, butyrate, pentanoate, hexanoate, heptanoate, levulinate, halides (such as chloride, bromide, iodide), citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate. It is known in the art that halide salts are in the form of acid halide for many of such salts (e.g., hydrochloride). The more preferred salt is hydrochloride. Such salts can become ionized in aqueous environment and the cation can be delivered to produce physiological effect on the patient. For example, fentanyl salt will form fentanyl cation and sufentanil will form sufentanil cation.

Especially useful narcotic analgesics that have cations are fentanyl hydrochloride, sufentanil hydrochloride and sufentanil citrate.

The rate of delivery of fentanyl (i.e., fentanyl HCl) and sufentanil (i.e., sufentanil HCl or sufentanil citrate) have been investigated and described before, e.g., in U.S. Pat. No. 6,216,033, and the method and rate of delivery (i.e., the current and flux) of such description can be adapted for the present invention. Briefly, for fentanyl HCl, the transdermal electrotransport flux remains independent of fentanyl HCl concentration at or above about 11 to 16 mM on solvent substantially throughout the fentanyl ion electrotransport delivery period. By maintaining the concentration of fentanyl HCl solution at or above about 11 to 16 mM in the donor reservoir, the electrotransport flux of the drug remains substantially independent of the drug concentration in the donor reservoir solution and substantially proportional to the level of electrotransport current applied by the delivery device during the electrotransport drug delivery. Maintaining the fentanyl salt solution concentration above about 11 mM, and preferably above about 16 mM ensures a predictable fentanyl flux with a particular applied electrotransport current. Adequate fentanyl salt (e.g., fentanyl HCl) is loaded into the anodic reservoir before the device is used, e.g., for 1-day delivery. It is noted if fentanyl salts other than fentanyl HCl is used, the equivalent concentration can be calculated from the above.

It has been determined that a transdermal electrotransport dose of about 20 μg (microgram) to about 60 μg of fentanyl (base) equivalent, delivered over a delivery interval of up to about 20 minutes, is therapeutically effective in treating moderate-to-severe post-operative pain in human patients having body weights above about 35 kg. Preferably, the amount of fentanyl delivered is about 35 μg to about 45 μg over a delivery interval of about, 5 to 15 minutes, and most preferably the amount of fentanyl delivered is about 40 μg over a delivery interval of about 10 minutes. Since fentanyl has a relatively short distribution half life once delivered into a human body (i.e., about 3 hours), the method of inducing analgesia preferably includes a method for maintaining the analgesia so induced. Thus the method of transdermally delivering fentanyl by electrotransport preferably includes delivering at least 1 additional, more preferably about 10 to 100 additional, and most preferably about 20 to 80 additional, like dose(s) of fentanyl over subsequent like delivery interval(s) over a 24 hour period. A current of about 150 μA to about 240 μA can be used. Adequate fentanyl salt (e.g., fentanyl HCl) is loaded into the anodic reservoir before the device is used, e.g., for 1 day or multiple day delivery (e.g., 2 days, 3 days, etc.).

The fentanyl HCl loading in the IONSYS fentanyl delivery system is about 10.8 mg fentanyl free base equivalent in 600 mg PVOH gel for delivery of about 3.2 mg fentanyl free base equivalent maximum. Generally a drug delivery device is approved by a competent national drug administration authority rated for a maximum delivery amount. For example, the IONSYS system was authorized by the USFDA to deliver a maximum of 80 doses of 40 μg per dose. Thus, the IONSYS system was designed and approved by drug administration authority to deliver a maximum amount of 3200 μg of fentanyl base equivalent. The IONSYS system can be said to have a nominal maximum delivery of 3200 μg of fentanyl base equivalent. However, in the present invention, with the incorporation of anion source in the anodic electrode, the amount of cationic drug loading can be reduced and still deliver the amount of the drug for which the device is designed and approved and prevent epithelial discoloration due to silver migration to the skin. Preferably, the amount of drug (e.g., fentanyl HCl) loading in the anodic reservoir is less than double the amount of drug the system is designed to deliver at a maximum. For example, if the device is designed to deliver 3200 μg of fentanyl at maximum, the device contains less than 6400 μg of fentanyl (correspondingly the equivalent amount of fentanyl HCl) and still does not cause skin staining. At the end of the delivery of a maximum amount of the drug, the drug remaining in the anodic reservoir is preferably 50% or less, preferably less than 50%, more preferably 40% or less, even more preferably 30% or less of the drug amount originally present in the electrotransport system at the start. Thus, although more fentanyl loading can be used, preferably, to reduce fentanyl abuse risk, fentanyl loading is 200% or less of the maximum amount of fentanyl designed to be delivered by the device. We have shown that using the composite electrodes of the present invention we were able to use fentanyl loading about 60% that of the IONSYS system and still achieve comparable prevention of skin staining. Thus, systems with fentanyl loading of about 6.4 mg fentanyl base equivalent loading to deliver nominal amount of 3.2 mg fentanyl base equivalent can be done. Therefore the electrotransport system of the present invention poses a smaller risk of being abused.

For sufentanil, preferably the sufentanil content is such that it is above a level to allow the flux to be independent of the sufentanil concentration. The transdermal electrotransport flux remains independent of sufentanil concentration at or above about 1.7 mM substantially throughout the sufentanil electrotransport delivery period. By maintaining the concentration of sufentanil solution at or above about 1.7 mM in the donor reservoir, the electrotransport flux of the drug remains substantially independent of the drug concentration in the donor reservoir solution and substantially proportional to the level of electrotransport current applied by the delivery device during the electrotransport drug delivery. Maintaining the sufentanil solution concentration above about 1.7 mM sufentanil equivalent ensures a predictable sufentanil flux with a particular applied electrotransport current.

Adequate sufentanil salt (e.g., sufentanil HCl) is loaded into the anodic reservoir before the device is used, e.g., for 1 day or multiple day delivery (e.g., 2 days, 3 days, etc.). A sufentanil dose of 2 μg to 12 μg (microgram or mcg) sufentanil base equivalent is therapeutically effective in treating moderate to severe post-operative pain in human patients having body weights above about 35 kg. Such a dose can be delivered over a delivery interval of up to about 20 minutes, such as 5, 10, 15 minutes, etc. Preferably the dose is 3.5 to 9 μg and most preferably about 5 to 7 μg, e.g., 6.5 μg. The sufentanil loading is adequate for delivery of such doses, preferably at or above about 1.7 mM during the period of delivery, of 1 to 3 days. For example, doses can be administered for 10 minutes per dose, up to 6 doses per hour.

Since sufentanil has a relatively short distribution half life once delivered into a human body (i.e., about 3 hours), the method of inducing analgesia preferably includes a method for maintaining the analgesia so induced. Thus the method of transdermally delivering sufentanil by electrotransport preferably includes delivering at least 1 additional, more preferably about 10 to 100 additional, and most preferably about 20 to 80 additional, like dose(s) of sufentanil over subsequent like delivery interval(s) over a 24 hour period. A current of about 50 μA (microAmp) to about 100 μA can be used. Since the chemistry of precipitation of metal halide, e.g., silver chloride is the same for fentanyl, sufentanil, or other fentanyl analogs, or other cationic drugs, the anodic electrode of the present invention would function similarly in the electrotransport delivery of other cationic drugs, such as cations of other narcotic opioid fentanyl analogs or normarcotic drugs. With an electrode with a built-in chloride source, it is understood by one skilled in the art that any cationic drug (not limited to fentanyl analogs) that can be delivered by electrotransport can be delivered using the composite electrode of the present invention.

Incorporation of the drug solution into the gel matrix in a reservoir can be done in any number of ways, i.e., by imbibing the solution into the reservoir matrix, by admixing the drug solution with the matrix material prior to hydrogel formation, or the like. In additional embodiments, the drug reservoir may optionally contain additional components, such as additives, permeation enhancers, stabilizers, dyes, diluents, plasticizer, tackifying agent, pigments, carriers, inert fillers, antioxidants, excipients, gelling agents, anti-irritants, vasoconstrictors and other materials as are generally known to the transdermal art. Such materials can be included by on skilled in the art.

The eletrotransport devices of the present invention can be included in a kit that contains the device and includes an instruction print, such as an insert or printings on a container, and the like, that provides instruction on the how the device is to be applied to a patient and how often the device can be activated and the maximum amount of drug the device is designed to deliver, etc. The instruction of use can include a method of activating the device and determining the doses and amount of drug already delivered. The instruction of use can also include brief description of the drug, the construction of the device, pharmacokinetic information, information on disposing the device that contains a control substance (e.g., fentanyl) and warnings.

### Biocompatibility of SEPHADEX™ Resin

In electrotransport in which a drug reservoir is in contact with the body surface, e.g., skin, for hours, e.g., 20 hours, 24 hours, or more, it is important that the material in the drug reservoir is biocompatible with the body surface, e.g., skin. Certain reservoir carrier matrix material such as PVOH has been shown to be biocompatible in the art and is already used in iontophoretic devices. However, suitable biocompatible anion exchanger has not been found, especially for strong anion exchanger. We have found that dextran-based strong anion exchanger resins, such as SEPHADEX™ QAE resin, to be biocompatible, in that the extracts of such resins do not cause adverse reaction in skin, and therefore would not be expected to cause inflammation, erythema or edema when anode electrodes with such resins are used with reservoirs deployed on skin for electrotransport. Inflammation, erythema or edema can be considered to cause discoloration of skin since they cause abnormal appearance, especially in color on the skin.

SEPHADEX™ QAE A-25 resin was extracted with four extraction vehicles: 1) 0.9 wt % sodium chloride USP solution (SC); 2) ethanol in saline 1:20 solution (AS); 3) polyethylene glycol 400 (PEG); and 4) cottonseed oil, NF (CSO). The extractions were made at a ratio of 2 g resin to 20 ml vehicle at 50° C. for 72 hours with pH adjusted to 7 with sodium hydroxide if necessary. The resin particles were filtered off to obtain the extracts. Mice were weighed and five mice were each injected either intravenously or intraperitoneally with each test extract at a dose of 50 ml/kg of extract (SC, AS, or CSO) or 10 g/kg of PEG extract. The corresponding extraction vehicles without extracting from the ion exchanger were also injected into control mice as controls. For PEG, the PEG extracts and control blanks were diluted with saline to make 0.2 g of PEG/ml, which corresponded to injection volume of 50 ml/kg. The mice were observed for adverse reactions such as convulsions or prostration, weight loss or death. The result showed that weight data were acceptable, there was no mortality, and the mice injected with the extracts appeared normal, without unexpected events. The ones injected with AS extracts appeared similar to those in the AS control as there may be lethargic effect caused by ethanol from the vehicle. Therefore, there was no evidence of toxicity with the test extracts.

SEPHADEX™ QAE A-25 extracts for SC, AS, PEG and cottonseed oil were used at 2 g ion exchanger to 20 ml vehicle similar to the above. The PEG extracts and control blanks were diluted with SC vehicle to make 0.12 g of PEG/ml. New Zealand white female rabbits were tested with intracutaneous injection with the extracts and controls. Each test rabbit was injected with 0.2 ml of test extract or the corresponding vehicle. Observation for erythema (ER) was conducted for 72 hours with rating scale of 0 to 4, wherein 0 means no sign of erythema, 1 means barely perceptible color change, 2 means a well defined pink color, 3 means moderate to sever redness, and 4 means severe redness (beet red) to slight eschar formation. Observation for edema (ED) was conducted for 72 hours with rating scale of 0 to 4, wherein 0 means no sign of edema, 1 means barely perceptible edema, 2 means a slight well defined area of swelling, 3 means moderate edema with raised about 1 mm, and 4 means severe edema (raised more than 1 mm and may extend beyond the area of exposure). The result showed that for SC, AS, and PEG the ED and ER were all 0. For the CSO extracts, the extract results and control results were the same, with a score of 2 for ER and a score of 1 for ED. Thus, the rabbit ER and ED tests showed that SEPHADEX™ QAE was biocompatibility and would not cause ER, ED or skin physiological color change due to inflammation in the skin (in other words, discoloration due to such skin changes).

Further, test results of the effect of test extract in vitro on lymphocyte proliferation (stimulation index) and cytotoxicity (IC₅₀) on HELA cells showed that SEPHADEX™ QAE resins are nontoxic and nonmitogenic. Extracts of ion exchange resins were generated from powder based polymers under passive (aqueous) conditions. The materials were examined for their mitogenic and cytotoxic activities. Mitogenicity tests were performed using in vitro lymphocyte proliferation assays. Cell cytotoxicity was assessed using MTT and LDH release assays. Mitogenicity testing was performed on lymphocytes obtained from mice, guinea pig, rat, and humans. Human fibroblasts and HELA cells were used for cytotoxicity testing. Cholestyramine resin (C1734 Cholestyramine Resin, USP from Spectrum Chemicals, Gardena, Calif., USA) was also tested similarly for comparison.

### Preparation of Passive Resin Extracts

Ten mL of RPMI-1640 culture medium (containing penicillin/streptomycin) was added to one gram of the test resin (dry form) and placed in a 50 mL conical tube. The solution was placed on a circulating rotator (slow speed rotator) for 72 hours at room temperature. Thereafter, extracts were obtained by centrifuged at 500 g (10 min). The supernatant was collected and sterile filtered through 0.22 μM filter and stored frozen (−20° C.) as extract till use. The remaining pellet was discarded. These extracts were tested for biocompatibility by tests for mitogenic activity with lymphocytes and on cytotoxicity.

Isolation of Lymphocytes from Mouse Spleen or Lymph Nodes

Lymph nodes (axillary, brachial, inguinal, popliteal, and cervical) and/or spleens from euthanized animals were removed under aseptic conditions and placed in sterile tube containing PBS, or similar media. The tissues were then teased to release the cells. Cells were filtered, centrifuged, washed and separated with standard procedures known in the art to separate lymphocytes. Cell counts were determined using a hemocytometer and viability was assessed using trypan blue. The cells were resuspended to a final concentration of 2-3×10⁵ cells/mL (10% FBS final concentration in culture).

Isolation of Lymphocytes from Rat or Guinea Pig Spleen

Spleens from euthanized animals were removed under aseptic conditions and placed in sterile tube containing PBS, or similar media. The tissues were then transferred into a sterile Petri dish containing cell culture media. Cells were released by teasing the tissue cells with forceps and syringe/needle. Cells were filtered, centrifuged, washed, over Lympholyte-M (room temperature), and separated with standard procedures known in the art to separate the lymphocytes with procedures known in the art. Cell counts were determined using a hemocytometer and viability was assessed using trypan blue. The cells were resuspended in culture medium to a final concentration of 2-3×10⁵ cells/mL.

Isolation of Guinea Pig Lymphocytes from Peripheral Blood

Blood was collected from guinea pigs under sterile conditions into sodium citrate tubes. Blood was diluted 1:1 with 1×DPBS (1% penicillin/streptomycin) into sterile polypropylene tubes. The cells were then layered blood over Lympholyte-M (room temperature) and separated out the lymphocyte cells with procedures known in the art and similar to the above.

Isolation of Human Lymphocytes from Peripheral Blood

Human blood was collected under aseptic conditions by venipuncture into sterile heparinized tubes. The blood was transferred to sterile 50 mL polypropylene tubes and diluted 1:1 with 1×DPBS containing 1% penicillin/streptomycin. The diluted sample was carefully layered Histopaque-1077 separation media (adjusted to room temperature). The samples were then centrifuged for 20 minutes at 400 g. After centrifugation, the lymphocytes were collected at the interface and transferred to 50 mL tubes. The suspension was adjusted to about 35-40 mL with 1×DPBS with 0.1% BSA (adjusted to 4° C.) and centrifuge at 400 g for 10 minutes. The supernatant was discarded. Removal of residual red blood cells present in the pellet was accomplished by the addition of 4.5 mL of sterile deionized water and resuspension of the cells. Shortly thereafter, 0.5 mL of 10×DPBS was added in order to restore isotonic conditions. Culture medium was then added. The cells were resuspended to 3.0×10⁶ cells/mL in RPMI cell culture medium (final serum concentration in culture is 5% NHS).

### Lymphocyte Proliferation Assay

For each sample, 100 μL of PBL (3.0×10⁶ cells/mL) were dispensed into a 96-well round-bottom plate (3.0×10⁵ cells/well). To this, 100 μL of media containing appropriate reagents (e.g., test antigens or controls), i.e., extract, were added to bring the final volume to 200 μL/well (depending on cell type, either 10% FBS or 5% NRS or 5% NHS). Replicate wells (at least triplicates) were established for each variable. Cells were maintained in a tissue culture incubator (37° C., 5% CO₂). Twenty four hours after culture initiation (day 1), the cell were pulsed with 1 μCi of ³H-thymidine (20 μl/well, 50 μCi/mL stock). On Day 2 (18-24 h after pulse), cells were harvested using cell harvester (Packard GF/C plates). After harvesting, GF/C filter plates were allowed to air-dry. The underside of the GF/C plates were sealed with an adhesive, and 20.5 μl of MicroScint-20 is added to each well. A seal (TopSeal) was placed on to cover the top of plate. ³H-thymidine incorporation was determined by β scintillation counting (Packard TopCount). Cultures were evaluated for 48 to 72 hours. As a measure of cellular proliferation, the results were expressed in counts per minute (CPM). Each variable was evaluated in at least triplicates, and the results were calculated as average CPM +/−the standard error of the mean (SEM). Lymphocyte proliferative responses to the test compounds were compared to cell cultured in media alone (i.e. background). The data were also expressed as stimulation index (SI) and were calculated from:

$$SI = \frac{average C P M for stimulated wells}{averages C P M from unstimulated control wells}$$

A response is considered positive if the SI value is >2.0, and the response is dose dependent.

### MTT and LDH Cytotoxicity Assays

In the assays, suspensions of 2.0×10⁴ cells were added per well in a flat-bottomed 96-well plate. Cells were allowed to adhere to the plate overnight. Thereafter, the media was removed, and 200 μL of test solution (i.e., resin extract) was added per well. Test solutions were incubated with cells for 20 hours. After incubation, the supernatants were collected and used for LDH release (Lactate Dehydrogenase Release) assay. The MTT assay ((3-[4,5-dimethylthiazol-2-yl]-2,5-diphenyl tetrazolium bromide) assay) was performed on the adherent cells. MTT assay and LDH release assay are well known in the art of cytotoxicity evaluation.

### Results

MTT and LDH release assays were performed for each of the extracts obtained above. SEPHADEX™ QAE showed no cytotoxicity. In contrast, USP grade cholestyramine resin showed cytotoxicity because the 50% inhibitory concentration for (IC₅₀) cholestyramine was found to be at a 1:18.5 dilution. There was no mitogenic activity in lymphocytes cultured with SEPHADEX™ QAE. There was no significant mitogenic activity with SEPHADEX™ QAE in any of the tests. Mouse (strain: Balb/c) lymphocytes demonstrated a positive lymphocyte response to cholestyramine (stimulation index=14-33). Guinea pig lymphocytes, isolated from peripheral blood or spleen, showed no reactivity to cholestyramine. Rat lymphocytes, derived from spleen cells, showed positive lymphocyte activity towards cholestyramine (stimulation index=3.9). Human peripheral blood mononuclear cells (PBMC) showed no activity towards cholestyramine.

Also, tests to show histamine release from mouse mast cells (cell line 10P2) showed that when the cells were cultured with SEPHADEX™ QAE A-25 resin extract there was no increase in histamine release. Thus, all the evidence indicated that SEPHADEX™ QAE resin caused no adverse biocompatibility reaction at all. From our experimental results we found that the SEPHADEX™ QAE strong anion exchanger is exceptionally biocompatible, considering that we have found even USP grade cholestyramine resin is not as biocompatible as the SEPHADEX™ QAE ion exchanger.

### EXAMPLES

First, methods for making electrode are illustrated by a composition containing silver, SEPHADEX™ QAE A-25, Poly(vinylidene fluoride) (PVDF), and N-Methylpyrrolidone (NMP). The particles (silver and SEPHADEX™ anion exchanger and the solvent and PVDF were used as received and were not dried prior to processing).
1. PVDF (about 0.5 million Da MW) was dissolved in NMP completely till a transparent solution was obtained.
2. Ag flakes were mixed with SEPHADEX™ QAE A-25 beads.
3. The mixture of step 2 was added to the solution of step 1.
4. The composition was mixed in mixing equipment till a grayish slurry was obtained. The ingredients were dispersed in the mixture. The relative amount of the ingredients were: Ag flakes 34 wt %, SEPHADEX™ QAE A-25 beads 6 wt %, PVDF 6 wt % for a total of 46 wt %; NMP the balance, which was 54 wt %.
5. The slurry was cast on an electrically conducting adhesive tape (E-CAT) or a release liner (either using a doctor blade or similar equipments or controlled by weight). The slurry cast on E-CAT was put into a forced air oven. The electrode was dried in a forced air oven at 100° C. till the NMP evaporated.
6. Once dried, the electrode was stored in a pouch free from moisture.

Temperature and humidity for steps 1-5: Room Temperature (21° C.). Humidity: about 35%.

For a process of forming a laminate, the slurry of step 5 was cast on a release liner instead of E-CAT and dried. The resulting cast material was laminated with E-CAT. Anode electrode of cast material on E-CAT was used for testing drug flux. For ½ inch (1.27 cm) diameter with an area of 1.27 cm² electrode, 0.0204 g of Ag was used, which was equivalent to 1 mil thick Ag foil. The thickness of the slurry in step 5 was adjusted depending on the formulations. When mesh was used, mesh was placed on the ECAT. The composition of the slurry was 5 wt % Ag flakes, 45 wt % SEPHADEX™ QAE A-25 beads, 5 wt % PVDF, and 45 wt % NMP. The slurry was cast on the mesh. When foil was used, foil was placed on the ECAT. The composition of the slurry was 10 wt % Ag flakes 40 wt % SEPHADEX™ QAE A-25 beads, 5 wt % PVDF, and 45 wt % NMP. The slurry was cast on the foil to from a composite electrode with silver particles and silver foil. The composite electrodes were made to contain an adequate amount of silver so that the amount of silver was not the limiting factor in the flux as time progressed and silver was consumed. In these cases, since the current was controlled, the flux change with time was mainly affected by the fentanyl content remaining in the reservoir on which the electrode was applied. In the following experiments, when different electrodes were tested and compared, the anodic electrode made with a silver foil and silver particles contained a silver foil similar to the silver foil in the control silver foil electrode.

### Example 1

### In vitro Experiments of Fentanyl HCl Flux

In vitro iontophoretic experiments were done with heat separated human epidermis.

Custom-built DELRON horizontal diffusion cells made in-house were used for all in vitro skin flux experiments. The process was generally as follows. Anode with the same polarity as the drug is adhered to one end of the cell that functions as the donor cell. The counter electrode made of AgCl is adhered at the opposite end. These electrodes are connected to a current generator (Maccor) that applies a direct current across the cell. The Maccor unit is a device with in-built compliance voltage up to 20 V to maintain constant iontophoretic current. For all in vitro electrotransport experiment, heat separated human epidermis is used. In a typical experiment, the epidermis is punched out into suitable circle ( 15/16 in, i.e., 2.4 cm) and refrigerated just prior to use. The skin is placed on a screen ( 15/16 in) that fits into the midsection of the DELRON housing assembly. Underneath the screen is a small reservoir that is 0.5 in (1.25 cm) in diameter, 1/16 in (0.16 cm) deep and can hold approximately 250 μl (mcl) of receptor solution. The stratum corneum side of the skin is placed facing the drug containing hydrogel. The receptor solution (saline, phosphate or other buffered solutions compatible with the drug) is continuously pumped through the reservoir via polymer tubing (Upchurch Scientific) connected to the end of a syringe/pump assembly. The pump can be set to any desired flow rate. The drug containing polymer layer, is placed between the donor electrode and heat separated epidermis. A custom-built DELRON spacer is used to encase the drug layer such that when the entire assembly is assembled together, the drug-containing polymer is not pressed against the skin too hard as to puncture it. A number of spacers of varying thicknesses can be placed together using double-sided adhesives to accommodate polymer films of varying thicknesses or even multiple films. Double-sided adhesive is used to create a seal between all the DELRON parts and to ensure there are no leaks during the experiment. The entire assembly is placed between two heating blocks that are set at 34° C. to replicate skin temperature. The receptor solution is collected by the collection system, Hanson Research MICROETTE, interfaced to the experimental set up. The samples are collected from the reservoir underneath the skin directly into HPLC vials. The collection system is programmed to collect samples at specified time intervals depending on the length of the experiment, for example, at every hour for 24 hours. The Hanson system is designed such that it can collect from up to twelve cells. From the twelve cells, a piece of tubing takes the receptor solution to the MICROETTE and dispenses it into the HPLC vials loaded onto a rotating wheel that can hold up to 144 vials, or 12 vials for each cell. Once the vials on the wheel are filled, the vials can be replaced with empty vials to collect more samples. The samples can then be analyzed via HPLC to determine delivery efficiency of the drug in the formulation. A 1/10 diluted Delbeccos phosphate buffered saline (DPBS) receptor solution has been used as the receiver fluid in vitro since it showed a good correlation of in vivo in vitro flux in the prior art. The buffer is pumped into the receptor solution reservoir at 1 ml/hr. The Hansen MICROETTE collection system was programmed to collect every 1½ hour for 16 intervals over a 24 hour delivery experiment. The receptor solution flow can also be adjusted to higher or lower values.

In each case, the drug was fentanyl hydrochloride at a concentration of 1.04 wt % in the drug-containing chamber. The anodic electrodes were made with silver flakes and anion exchange resin particles embedded in a PVDF binder and a solvent NMP. Another two kinds of electrodes (anodic electrodes with a silver mesh and particles with anion exchange resin particles; and anodic electrodes with silver foils and particles with anion exchange resin particles) were made and tested in comparison with control electrodes (which were anodic electrodes that merely included conventional silver electrode connected to the drug compartment). The mesh was purchased from Advent Research Materials Ltd. There were 198 wires/cm² and the purity was 99.99%. Aperture size was 0.44 mm-0.6 mm and open area was 53.23%. The foil with a thickness of 1 mil (0.025 mm) composed of 99.99% silver was purchased from Ames Electro Corp. The mesh and the foil were punched to ½″ diameter and placed on the ECAT. Various sizes of Ag particles and flakes were purchased from Sigma-Aldrich. Preferably 10 μm Ag flakes composed of 99.9+% silver was used. The PVDF binder with an average MW of 534,000 was purchased form Sigma-Aldrich. SEPHADEX™ QAE A-25 was purchased from Sigma-Aldrich and used as received. In laboratory processing, 2-10 g of slurry was made and approximately 30-90 mg of the slurry was cast on the ECAT. The foil and mesh were used for making electrodes in the examples below. However, it is to be understood that the above foil and mesh are illustrative of suitable material. The thickness of the silver foil and wire size of the mesh are not critical so long as they are adequate to remain in good condition after the electrotransport use.

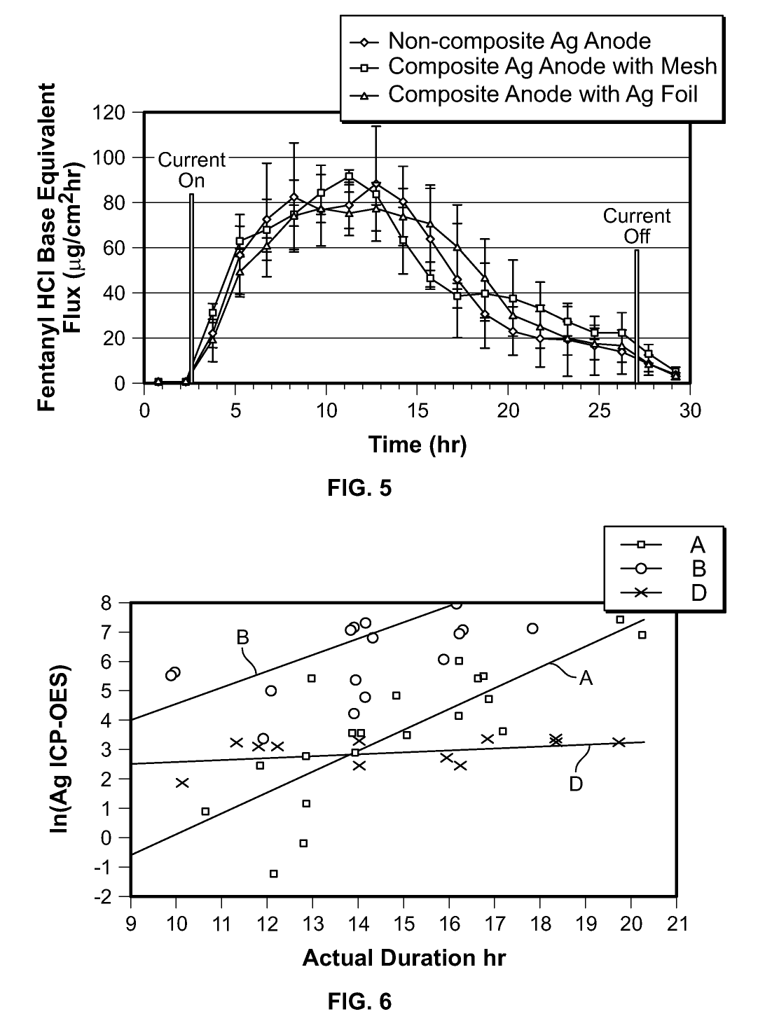

FIG. 5 shows that comparable delivery profile across heat separated human epidermis for the steady state flux and duration using composite anode for two different configurations namely foil/particle and mesh/particle. The current was applied at 100 μm/cm² for 24 h. Iontophoretic current was turned on at about 2.5 hour and turned off at about 27 hour. The composite anodes performed well similar to the control silver anode. The flux was high for up to about 15 hours. Thus, using the composite anode, we were able to deliver the drug at an acceptable flux. The anode with silver particles retained high flux for a little longer than the anode with silver mesh.

### Example 2

### In vitro Experiments

Another cationic drug in non-HCl form (normarcotic) with molecular weight higher than fentanyl HCl was delivered across heat separated human epidermis using the composite (silver mesh and particles) anodic electrodes at 100 μm/cm² for 24 h with systems like those in Example 1. To compare the performance of composite silver mesh and particle electrode, the control was run with a chloride source containing interface hydrogel placed between the Ag foil and the drug-containing reservoir. The interface gel contained 1.3 wt % SEPHADEX™ QAE A-25 in which the SEPHADEX™ was in chloride form. The result also showed that the composite (silver mesh and particles) anodic electrode, as well as the interface chloride source electrode, was able to deliver the drug at a flux that was quite stable over a period of about 24 hours. No silver staining on the skin was observed post flux. This showed that the composite chloride electrode can be used for delivery a normarcotic non-HCl drug.

### Example 3

### In vivo Experiments

Electrotransport delivery using composite anode was carried out on Yorkshire swine at 100 μm/cm² with formulations containing 40% lower drug loading than the IONSYS™ system, which had the fentanyl HCl drug loading of 1.74 wt %. The systems with composite anode showed no signs of silver migration on the skin or the skin side gel up to 20 hours at this current density of 100 μm/cm², which was about 64% higher than IONSYS™, fentanyl HCl delivery system. The electrosubstrate was made with the form the dimensions of which are outlined below. The electrosubstrate had two gel reservoirs, namely cathode and anode. The gel area was maintained at 1.27 cm² and the x-y dimensions of the substrate across the center were approximately 7 cm and 3.8 cm respectively. The configuration at the anode contained two layers of gel each with a thickness of 1.2 mm ( 3/64 inch). The gel closer to the anode and the gel closer to the skin were named the anode side gel and the skin side gel. The two-layer or split layer configuration was used to facilitate the removal of gels post flux to analyze Ag concentration in the skin side gel. Because the two layers are alike, the two-layer configuration does not affect the fentanyl delivery as compared to a single layer of a thickness equal to the two. The two layers added up to a thickness of about 2.4 mm, like that of a drug gel layer in prior fentanyl delivery devices, IONSYS™ system. The anode gel was PVOH based hydrogel containing 1.04 wt % of fentanyl HCl, 40% lower drug loading than the IONSYS™ system. The skin was dissected at the end of the study. The skin was analyzed for silver content using two methods, ICP: OES-Inductive coupled plasma-optical emission spectroscopy detector and ICP-MS-inductive coupled plasma-mass spectrometry detector. Such ICP: OES and ICP-MS analysis methods are known to those skilled in the art. For the composite anode configuration, the anode side gel and the skin side gel were analyzed together because the gels were hard to be separated due to water uptake by the composite electrode. The result showed that there was no significant silver migration into the skin. Silver (Ag) concentration in the skin side gel and skin increased exponentially with time for 100% drug loading control and 60% drug loading control. Thus Ag concentration was converted to ln scale to get a linear relationship with time.

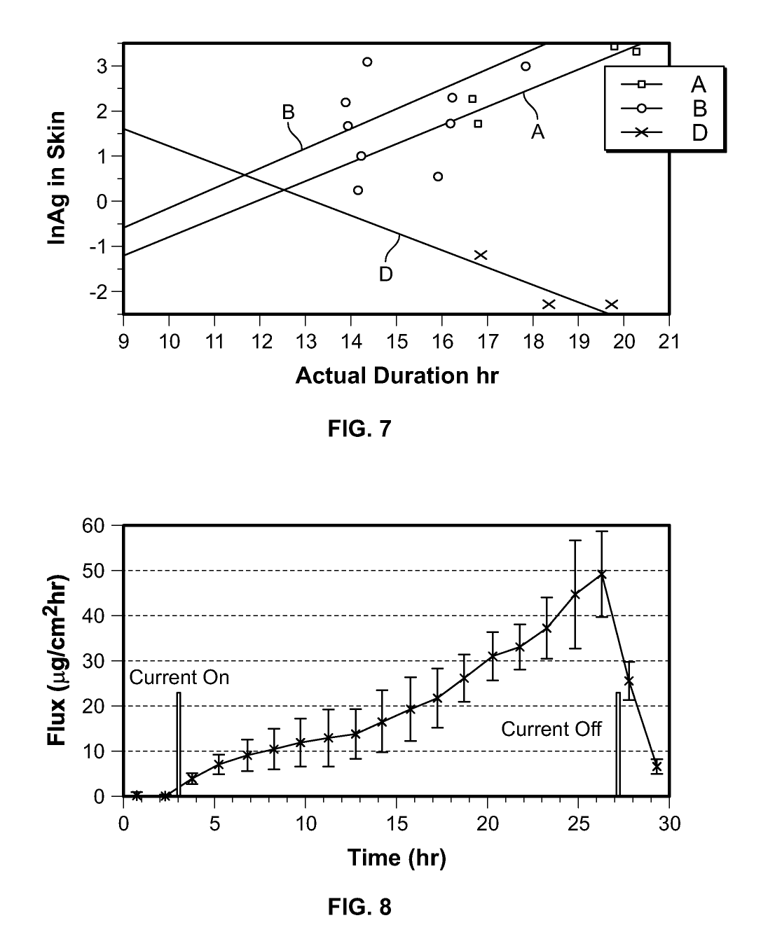

The graph in FIG. 6 shows the ln Ag (determined by ICP-OES) on the skin side gel as a function of duration for A (1.74 wt % Fentanyl HCl, 100% drug loaded control, i.e., silver electrode on reservoir with 100% of fentanyl hydrochloride drug loading as prior IONSYS™ device), B (silver electrode with 60% of drug loading as of the 100% drug loading control) and D (Composite anode with 60% drug loading as the 100% drug loading control). The amount of silver in the anode and skin side gel on the composite anodic electrode remained very stable as the results from the 9^{th} hour to the 20^{th} hour showed. No data was collected for the period earlier than the 9^{th} hour. There was no increase of silver in the skin side gel and the anode side gel for the composite anode as a function of duration of electrotransport. The control silver electrode associated with 100% drug loading reservoir showed data A having a gradually increasing silver content in the skin side gel. The silver electrode associated with 60% drug loading reservoir B also had gradually increasing silver content in the skin side gel. The silver content in the skin side gel for B (silver electrode associated with 60% drug loading reservoir) was higher than A and D. Similar results were obtained in the dissected skin, with which the silver content was determined using ICP-MS. The results were shown in graphical form in FIG. 7. There was no increase of silver in the skin for the composite anode (D) as a function of duration of electrotransport. The control silver electrode associated with 100% drug loading A had a gradually increasing Ag content in the skin samples. The silver electrode associated with 60% drug loading B also had a gradually increasing silver content in the skin samples. The silver content in the skin samples for B (silver electrode on 60% drug loading reservoir) was higher than A and D (composite anode).

The skin side gels and the skin for the above experiments were visually observed. Comparisons were made on silver staining in electrotransport comparing the use of composite electrodes with controls of using silver anode electrodes (in which some controls used 100% of fentanyl HCl drug loading in the drug reservoir, and other controls used 60% of fentanyl HCl drug loading in the drug reservoir). Silver staining was observed and scores were kept until after 48 hours after the electrotransport was finished to allow silver staining to develop discoloration on the skin. The scoring was defined from 0-4, i.e., 0 none, 1, negligible, 2 slight, 3 definite, and 4 dark. The results are shown in the following Table 1. The durations were the duration periods of iontophoretic delivery. The percentage in silver staining scores indicates the size of the silver staining relative to the anode size, 1.27 cm². N was the number of samples.

TABLE 1

| Formulation | Duration hr | N | Ag score |
|---|---|---|---|
| 100% control A | 11 h | 1 | 0 |
| | 12 h | 2 | 0 |
| | 13 h | 4 | 0 |
| | 14 h | 3 | 0 |
| | 15 h | 2 | 0 |
| | 16 h | 2 | 0 |
| | 17 h | 2 | 0 |
| | 18 h | 1 | 3, 1-25% |
| | | 1 | 1, 1-25% |
| 60% control | 9 | 1 | 1, 1-25% |
| | 10 | 1 | 0 |
| | 12 | 1 | 2, 26-50% |
| | 14 | 1 | 4, 26-50% |
| | | 1 | 1, 1-25% |
| | | 1 | 3, 1-25% |
| | 16 | 1 | 3, 1-25% |
| 60% with composite | 10 | 1 | 0 |
| anode | 11 | 1 | 0 |
| | 12 | 2 | 0 |
| | 14 | 2 | 0 |
| | 16 | 2 | 0 |
| | 17 | 1 | 0 |
| | 18 | 2 | 0 |
| | 20 | 1 | 0 |

The composite anodic electrodes were used on drug reservoirs with 60% fentanyl loading (i.e., 60% compared to that in the control silver foil electrode reservoir of 1.74 wt % fentanyl HCl). The skin on which the composite anodic electrodes were used for electrotransport, although used on reservoirs of 60% fentanyl HCl loading, did not show any observable silver stain up to 20 h of electrotransport. For the drug reservoirs on which the composite anodic electrodes were used, silver concentration was measured both in the anode side gel and the skin side gel, because two layers of gels were not easily separated. Even though the anode side gel was analyzed, the gels on which the composite anodic electrodes were used did not show any observable silver stain up to 20 h of electrotransport. There was no noticeable skin silver staining by visual observation where the composite was used. In contrast, the skin and the skin side gels on which the control electrodes associated with 60% fentanyl HCl loading were used showed silver stain in the skin side gel beyond 9 h and in the skin beyond 12 h of electrotransport. We have known from work in the past that excess amount of fentanyl HCl is needed to reduce silver staining in the skin in electrotansport. Thus, it is not surprising that control electrodes on reservoirs having 60% fentanyl HCl loading showed more silver staining than the control electrodes on reservoir with 100% fentanyl HCl loading. In contrast, we were able to achieve a result with no observable silver staining in the skin and in the skin side gel using the composite electrode, even with only 60% fentanyl HCl loading in the drug reservoir.

### Example 4

Fentanyl citrate was delivered with the composite anode at 100 μA/cm² for 24 h with process and set up similar to the above. The iontophoretic delivery current was turned on at hour 3 and turned off at hour 27. The result in FIG. 8 shows that the composite anodic electrode (silver mesh and particles) was able to deliver the drug at flux about 21 μg/(cm².hr) over 24 h on average. No silver staining on the skin was observed post flux. Thus, the composite anode was shown to be useful in delivery of non-HCl fentanyl drug.

### Example 5

Composite anodic electrodes were made with compositions having the formulations shown in the following Table 2. Ag is silver, Seph is SEPHADEX™ QAE A-25, and PVDF+NMP is a solution of the binder PVDF in solvent NMP. The numerical values are the fractional ratios of these three types of ingredients for ten formulations.

TABLE 2

| Formulation | Ag | Seph | PVDF + NMP |
|---|---|---|---|
| 1 | 0.42 | 0.18 | 0.4 |
| 2 | 0.51 | 0.09 | 0.4 |
| 3 | 0.6 | 0 | 0.4 |
| 4 | 0.35 | 0.18 | 0.47 |
| 5 | 0.5 | 0 | 0.5 |
| 6 | 0.40 | 0.10 | 0.51 |
| 7 | 0.28 | 0.18 | 0.54 |
| 8 | 0.28 | 0.12 | 0.6 |
| 9 | 0.34 | 0.06 | 0.6 |
| 10 | 0.4 | 0 | 0.6 |

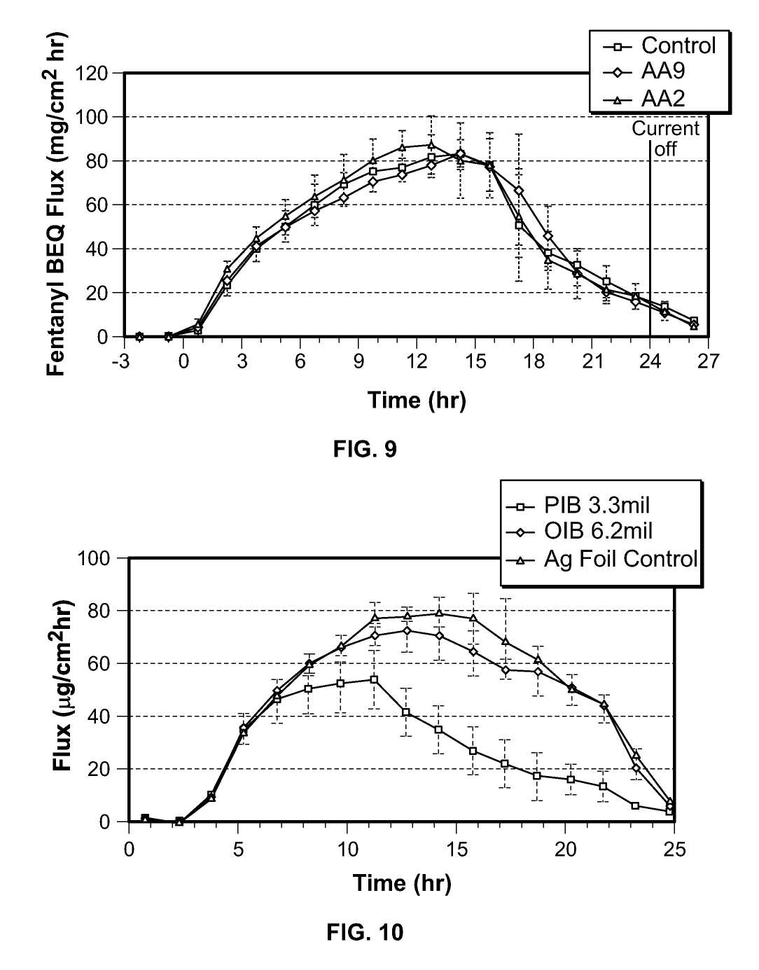

The composite electrodes with the formulations of Table 2 were made with the slurry casting on ECAT described above without silver mesh or silver foil. The amount of silver in the composite anodic electrodes was about equal to that in the control silver electrode which had a silver foil of 1 mil (0.025 mm) thick. The electrodes were made with silver flakes, SEPHADEX™ QAE A-25 particles and binder solution consisting of 10 wt % PVDF and 90 wt % of NMP. Electrical current was on from time 0 hr to 24 hr. The resulting anodic electrodes were tested for electrotransport with fentanyl HCl with fentanyl HCl (reservoir fentanyl HCl loading was 60% of the loading in the reservoir for the control silver foil electrode) versus a silver foil electrode similar to Example 3 on heat separated cadaver epidermis. For illustration, FIG. 9 shows the fentanyl base equivalent flux of the silver foil control electrode and the composite electrode of Formulation 2 and Formulation 9. Iontophoretic current was turned on at about 0 hour and turned off at about 24 hour. In FIG. 9, the squares represent the data for the control electrode; the diamonds represent the data (designated AA9) for electrode with Formulation 9; and the triangles represent the data (designated AA2) for electrode with Formulation 2. It is noted all three electrodes had similar flux profiles over time. According to the silver staining scoring system of Example 3 above, the silver staining score on the gel due to silver migration was zero for Formulation 2 and 9 indicating there was no silver staining.

The above examples illustrate that composite anodic electrode made with metallic pieces (e.g., flakes) and anion exchanger particles with or without metallic mesh or foil can function well in supporting electrotransport without causing silver staining (due to silver migration) on a surface through which drug is delivered, even at reservoir fentanyl HCl loading of only 60% of the loading in the reservoir for the control silver foil electrode.

The Examples below illustrate the making and use of composite electrodes having composite coat with a PIB binder.

### Example 6

Anodic electrodes were made with chloride source and PIB binder. The PIB based electrodes were prepared by using two grades of PIB with different molecular weights. A low molecular weight (MW) PIB (VISTANEX LM-MS or OPPANOL B12) and a higher MW PIB (VISTANEX MM L-100 or OPPANOL B100) were used. The electrodes were prepared by first dissolving the binders (both low and high MW PIB) in heptane for a period of 8-10 hours (but can be as long as a few days for high MW PIB to dissolve) under slow rotation (700-1000 RPM) stirring. The silver flakes and SEPHADEX were added to the binder mix till a uniform suspension was obtained. The mix was then cast on to silver foil and the electrode was dried to remove the solvent. The final electrode composition (without the solvent) was 74 wt % Ag flakes: 13 wt % SEPHADEX: 13 wt % PIB. The composite electrodes were made to contain an adequate amount of silver so that the amount of silver was not the limiting factor in the flux. In these cases, since the current was controlled, the flux change with time was mainly affected by the fentanyl content remaining in the reservoir on which the electrode was applied.

A ratio of High MW to Low MW PIB of about 1:1 was used as the binder for binding the particles to silver foil. The PIB ratios were maintained at a level to prevent obtaining tacky films. For example, a ratio of 1:4 (High MW to Low MW) produced films that were tacky on a silver foil. Such tacky material also did not anchor the silver foil well and showed tendency to slip when pressed at an angle.

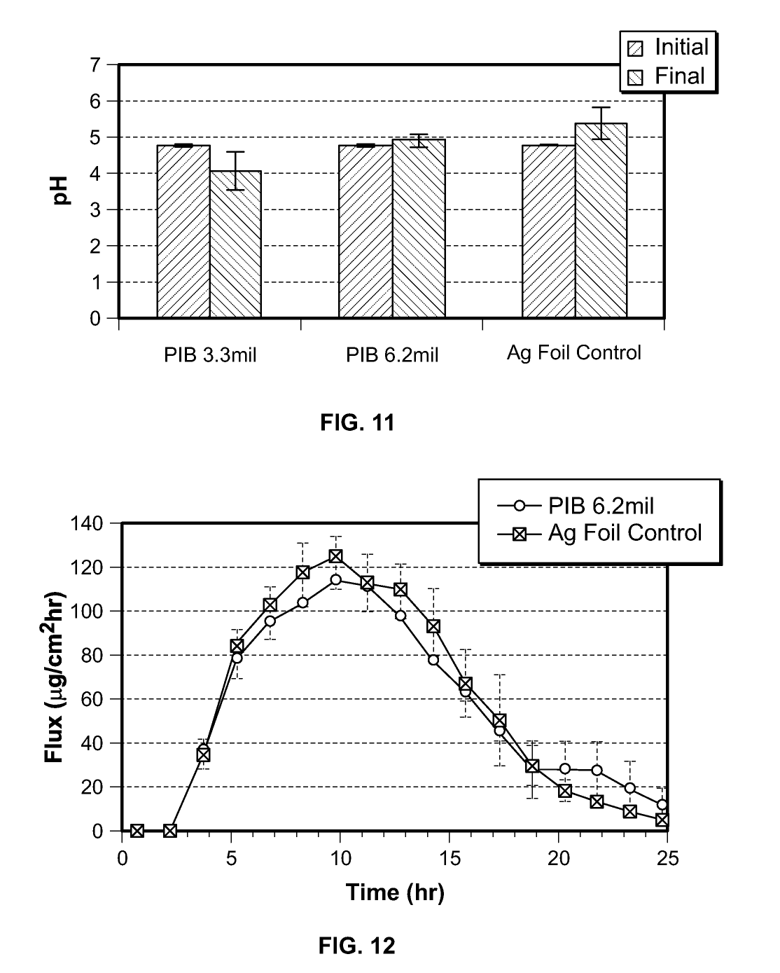

The electrodes were made such that the coating when dry had a thickness of 3.3 mil (0.083 mm) and 6.2 mils (0.155 mm). In vitro experiments were done with equipment process similar to those described in Example 1 above. FIG. 10 shows that comparable delivery profile across heat separated human epidermis for the steady state flux and duration using PIB composite anodes of two different thicknesses (i.e., 3.3 mil and 6.2 mils) and a control with 1 mil (0.025 mm) thick silver foil electrode. The reservoir for the control electrode had fentanyl hydrochloride loading 60% that of the IONSYS™ system (IONSYS™ system had about 1.75 wt % fentanyl HCl in fentanyl loading). The reservoirs for the two PIB electrodes also had fentanyl hydrochloride loading of 60% that of the IONSYS™ system (referred to as 60% fentanyl loading). Flux was determined for 3 hours of passive delivery without current and then 19.5 hours with an applied current of 100 μA/cm²; then another 3 hours of passive delivery at which the current was off (current was turned on at 3 hours and turned off at 19.5 hour). FIG. 11 shows the pH values at the initial stage and at the end of the experiment for each of the electrodes. For the 6.2 mil (0.155 mm) thickness electrode, the pH was very stable. But the 3.3 mil (0.083 mm) thickness electrode showed a decrease of about 0.8 pH units after the fentanyl transfer, compared to an increase of about 0.5 pH units in the control. As shown in FIG. 10, at a current density of 100 μA/cm², the electrode with a PIB composite coat thickness of 6.2 mils resulted in a flux profile with time similar to that of the control. The electrode with a PIB composite coat 3.3 mils thick resulted in a lower flux than the control through much of the 19.5 hour period of iontophoretic delivery. Thus, the PIB composite with the 6.2 mil thick coat was adequate to maintain the pH and steady state flux. Further, the silver staining result showed that there was insignificant silver staining in the skin and in the receiving side of the reservoir gel in the 6.2 mil thickness experiment. However, the control resulted in observable silver staining in the skin and in the gel on the receiving side of the skin. Thus, using the composite anode with 6.2 mil thick PIB composite coating, we were able to deliver the drug at an acceptable flux without staining the skin, or even part of the gel.

### Example 7

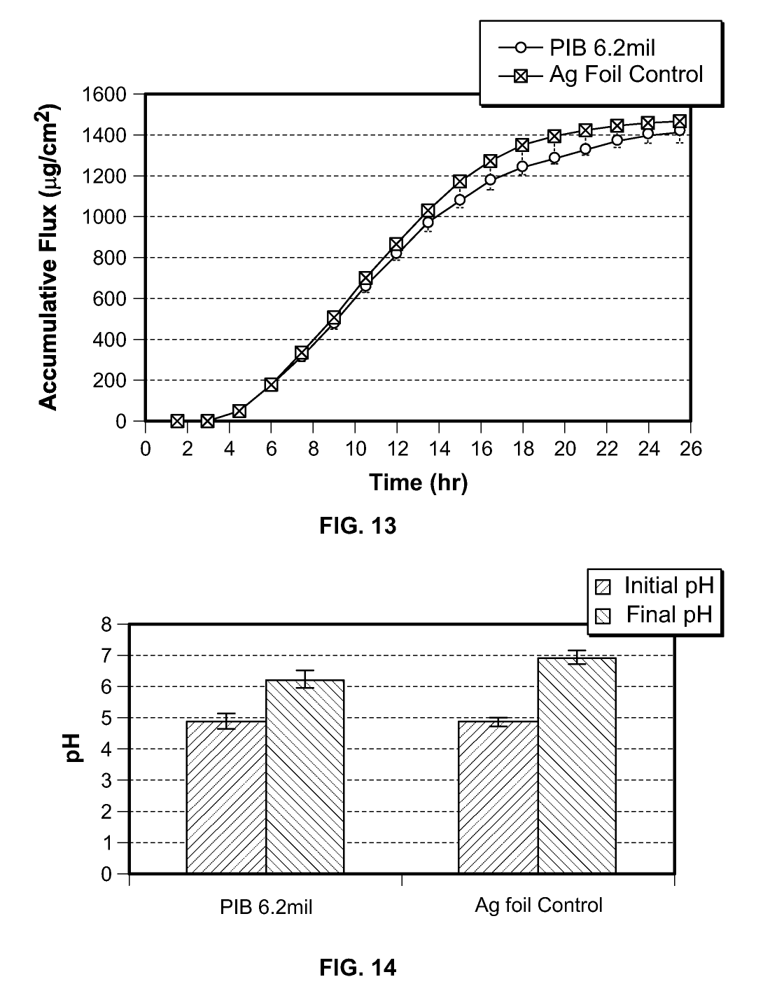

PIB composite electrode with a thickness of 6.2 mils like that of Example 6 was tested on skin from a donor different from that of Example 6 using equipment and process similar to that of Example 6. FIG. 12 shows the flux result of the PIB composite electrodes compared to that of a silver foil control electrodes for a 24 hour iontophoretic run. The curve with the circles data points represents the PIB data; the curve with the x data points represents the silver foil control data. The PIB composite electrodes were used on 60% fentanyl loading reservoirs and the silver foil control electrodes were used on reservoirs with 60% fentanyl loading. FIG. 12 shows that the PIB composite electrodes produced a fentanyl flux profile that was similar to that of the control electrodes. There was no significant silver staining in the runs with the PIB composite electrodes. However, the electrotransport of fentanyl with the control electrodes showed silver staining. FIG. 13 shows the accumulative fentanyl flux (in μg fentanyl base equivalent per cm²) as a function of time. Again, the PIB composite electrodes and the controls behaved similarly. FIG. 14 shows the pH shift of the experiments for the PIB composite electrode and the control silver foil electrode. Again, as in Example 6, the pH was very stable for the PIB composite electrodes, and appeared to be similar to the control silver foil electrodes.

It was further found that the inclusion of the silver foil in the PIB composite electrodes helped to further safeguard against moisture migration to the back the electrodes (farther away from the reservoir). We found that we could cast the PIB-containing composite slurry directly on a silver foil without any other adhesive material in between to form an anodic electrode and after drying the electrode would be sturdy and effective to enable cationic drug flux by electrotransport for at least a day.

The above-described exemplary embodiments are intended to be illustrative in all respects, rather than restrictive, of the present invention. It is to be understood that various combinations and permutations of various parts and components of the schemes disclosed herein can be implemented by one skilled in the art without departing from the scope of the present invention. The entire disclosure of each patent, patent application, and publication cited or described in this document is hereby incorporated herein by reference.

We claim:

1. An electrotransport system for iontophoretic administration of a drug through a body surface of a patient, comprising:
(a) anodic reservoir comprising a drug; and
(b) anodic electrode for conducting a current to drive the drug in the anodic reservoir in electrotransport, the anodic electrode having polymeric material with metal pieces and polysaccharide-based ion exchanger immobilized therein, the anion exchanger having precipitate-forming anions, the anodic electrode being disposed on a side of the anodic reservoir distal from the body surface, wherein the metal pieces generate metal ions during electrotransport and when the metal ions react with the precipitate-forming anions insoluble precipitate is formed in the polymeric material.

2. The system of claim 1 wherein the metal pieces are silver pieces, the precipitate forming anion is halide, and the polymeric material is in a polymeric layer form and the silver pieces are embedded in the polymeric material.

3. The system of claim 2 wherein the anion exchanger is dextran-based and the polymeric layer includes 30 wt % or more of silver particulates as silver pieces on dry basis.

4. The system of claim 2 wherein the layer of polymeric material is disposed on an electrically conductive adhesive in the anode electrode and interposes between the electrically conductive adhesive and the anodic reservoir.

5. The system of claim 2 wherein the layer of polymeric material has anion exchanger that is dextran-based and has tertiary or quaternary ammonium functionality, the anion exchanger being 5 wt % to 20 wt % dry basis of the polymeric layer.

6. The system of claim 2 wherein the layer of polymeric material has anion exchanger that is cross-linked dextran-based and has quaternary ammonium functionality.

7. The system of claim 2 wherein silver and anion exchanger are present at a ratio of silver to anion exchanger of 6:1 to 1:10.

8. The system of claim 2 wherein the anodic reservoir contains a hydrogel containing fentanyl hydrochloride and the system can deliver a flux of at least 60 μg/(cm² hr) fentanyl at 100 μA/cm² or more.

9. The system of claim 2 wherein the system can deliver drug effectively for at least 20 hours at 100 μA/cm² or more without staining the body surface and the system contains less than 200 wt % of the maximum amount of cationic drug the system is designed to deliver.

10. The system of claim 2 wherein the polymeric material includes particulate polymeric anion exchanger and a binder for binding the anion exchanger adjacent with the silver pieces.

11. The system of claim 10 wherein the polymeric material includes a hydrophobic fluorochemical binder for binding the anion exchanger and the silver pieces in the polymeric layer.

12. The system of claim 2 comprising polyvinylidene difluoride as binder for binding the metal pieces and the anion exchanger.

13. The system of claim 2 wherein the anion exchanger is cross-linked quaternary aminoethyl dextran with ionic capacity of 2.5-3.5 mmol/g on dry basis and containing quaternary ammonium functionality having chloride as the halide.

14. A method of making an electrotransport system for iontophoretic administration of a drug through a body surface of a patient, comprising:
providing anodic reservoir comprising the drug;
providing an anodic electrode made via solidifying a viscous composition having metal pieces, anion exchanger, and a polymeric binder to form an anodic electrode layer with anion exchanger and metal pieces immobilized by the polymeric binder, the anion exchanger being biocompatible polysaccharide-based anion exchanger having precipitate-forming anions, wherein the metal pieces generating metal ions in electrotransport and when the metal ions react with the precipitate-forming anions insoluble precipitates are formed in the anodic electrode layer; and
connecting the anodic electrode to a power source to provide electrical communication to the anodic reservoir for conducting electrical current to drive the drug from the anodic reservoir in electrotransport.

15. The method of claim 14 comprising connecting the anodic electrode on a side of the anodic reservoir distal to the body surface, the electrode layer includes anion exchanger particulates and 30 wt % or more silver particulates on dry basis, the anion exchanger contains halide ions and absorbs water when contacting a reservoir that contains water, and the silver particulates are embedded in the anodic electrode layer, and the halide ions being the precipitate forming anions, the method further comprising including a solvent for the binder in the composition.

16. The method of claim 15 comprising mixing the binder and the solvent to form a binder solution and mixing silver particles, polysaccharide-based anion exchange material and the binder solution to form the composition for forming the anodic electrode layer, the binder solution being 40 wt % to 60 wt % of the composition, the composition being a slurry.

17. The method of claim 15 comprising mixing silver particles, polysaccharide-based anion exchange material and 40 wt % to 60 wt % of a binder solution including the binder and solvent in the composition to form the electrode layer, wherein the binder is polyvinylidene difluouride (PVDF), the solvent is N-methyl pyrrolidone (NMP) or propylene carbonate at binder to solvent ratio of 1:20 to 1:10.

18. The method of claim 15 comprising mixing 20 wt % to 60 wt % of silver particles, 6 wt % to 18 wt % of cross-linked dextran-based strong anion exchange material and 40 wt % to 60 wt % of a binder solution containing the binder and the solvent to form a composition for forming the electrode layer, wherein the binder is polyvinylidene difluoride (PVDF), the solvent is N-methylpyrrolidone (NMP) or propylene carbonate at binder to solvent ratio of 1:20 to 1:10.

19. The method of claim 15 comprising mixing 20 wt % to 60 wt % of silver particles, 6 wt % to 18 wt % of tertiary or quaternary ammonium anion exchange material and 40 wt % to 60 wt % of a binder solution forming a composition and laying a layer of said composition to a substrate to form the electrode layer, wherein the binder is polyisobutylene; the binder solution containing the binder and the solvent.

20. The method of claim 15 comprising including in the anodic reservoir a hydrogel containing fentanyl hydrochloride such that the system can deliver a flux of at least 60 μg/(cm² hr) fentanyl at 100 μA/cm² or more.

21. A method of making an electrotransport system for iontophoretic administration of fentanyl ions through a body surface of a patient, comprising:
providing anodic reservoir comprising fentanyl hydrochloride ionizable into fentanyl ions;
making an anodic electrode having a polymeric layer including 10 wt % or more dextran-based quaternary ammonium anion exchanger particulates and 30 wt % or more silver pieces embedded in the polymeric layer, the anion exchanger particulates having precipitate-forming anions, wherein the silver pieces generating silver ions in electrotransport and when the silver ions react with the precipitate-forming anions insoluble precipitates are formed in the polymeric layer; the anodic electrode made via drying a composition having the silver pieces, anion exchanger particulates and a binder solution; and
connecting the anodic electrode to a power source to provide electrical communication to the anodic reservoir for conducting an electrical current to drive the fentanyl ions in the anodic reservoir in electrotransport, wherein there is no additional liquid containing layer more distal of the anodic electrode relative to the body surface, the system being capable of delivering therapeutic fentanyl ions for at least 10 hours without staining the body surface.

22. A method of drug electrotransport through a body surface of a patient without discolorizing the body surface, comprising:
placing a device for the iontophoretic delivery of drug on a patient, the device comprising anodic reservoir comprising a drug; and comprising anodic electrode for conducting a current to drive the drug in the anodic reservoir in electrotransport, the anodic electrode having polymeric layer with metal pieces and polysaccharide-based ion exchanger immobilized therein, the anion exchanger having precipitate-forming anions, the anodic electrode being disposed on a side of the anodic reservoir distal from the body surface, wherein the metal pieces generate metal ions during electrotransport and when the metal ions react with the precipitate-forming anions insoluble precipitate is formed in the polymeric layer; and
using the device to deliver the drug by electrotransport for at least 10 hours at 100 μA/cm² or more without staining the body surface.

23. A kit for administering a drug by electrotransport transdermally through a body surface of a patient, comprising:
(a) an iontophoretic device having anodic reservoir comprising a drug and having anodic electrode for conducting a current to drive the drug in the anodic reservoir in electrotransport, the anodic electrode having polymeric layer with metal pieces and polysaccharide-based ion exchanger immobilized therein, the anion exchanger having precipitate-forming anions, the anodic electrode being disposed on a side of the anodic reservoir distal from the body surface, wherein the metal pieces generate metal ions during electrotransport and when the metal ions react with the precipitate-forming anions insoluble precipitate is formed in the polymeric layer; and
(b) an instruction print including instruction on electrotransport delivery of the drug up to a maximum amount, wherein the maximum amount is more than 50% the drug contained in the device before use.

24. A method of preventing electrotransport discoloration of skin in iontophoretic delivery of a cationic drug, comprising:
applying an electrotransport device to the skin, the electrotransport device having
anodic reservoir comprising a drug and having anodic electrode for conducting a current to drive the drug in the anodic reservoir in electrotransport, the anodic electrode having polymeric layer with metal pieces and polysaccharide-based ion exchanger immobilized therein, the anion exchanger having precipitate-forming anions, the anodic electrode being disposed on a side of the anodic reservoir distal from the body surface, wherein the metal pieces generate metal ions during electrotransport and when the metal ions react with the precipitate-forming anions insoluble precipitate is formed in the polymeric layer; the device having a maximum delivery amount of the cationic drug designed to be delivered that is more than 50% of the amount originally present before use; and
using the device to deliver the cationic drug through the skin in an amount up to more than 50% of the amount originally present such that there is no observable discolorization on the skin.

25. The method of claim 24 wherein the electrode layer includes dextran-based ion exchanger particulates and 30 wt % or more silver particulates on dry basis, the anion exchanger contains chloride ions and absorbs water when contacting a reservoir, and the silver particulates are embedded in the polymeric layer which includes a polyvinylidene difluoride binder and the cationic drug in electrotransport is cationic fentanyl.

26. An electrotransport system for iontophoretic administration of a drug through a body surface of a patient, comprising:
(a) anodic reservoir comprising a drug; and
(b) anodic electrode for conducting a current to drive the drug in the anodic reservoir in electrotransport, the anodic electrode having polymeric material polysaccharide-based ion exchanger immobilized therein, the anion exchanger having precipitate-forming anions, the anodic electrode being disposed on a side of the anodic reservoir distal from the body surface, wherein metal ions are generated in the anodic electrode during electrotransport and when the metal ions react with the precipitate-forming anions insoluble precipitate is formed in the polymeric material.
